(19) United States
(12) Patent Application Publication
(10) Pub. No.: US 2026/0363654 A1
(43) Pub. Date: Nov. 19, 2026

(54) NEUROVASCULAR ULTRASONIC CATHETER, NEUROVASCULAR ULTRASONIC SYSTEM, AND A METHOD THEREOF

(71) Applicant: Sonox, Inc., Irvine, CA (US)

(72) Inventor: Carly Nicole Harad, Irvine, CA (US)

(73) Assignee: Sonox, Inc., Irvine, CA (US)

(21) Appl. No.: 19/211,144

(22) Filed: May 16, 2025

(51) Int. Cl. A61B 8/08; A61B 8/00

(52) U.S. Cl. CPC A61B 8/0891; A61B 8/0808; A61B 8/445; A61B 8/4488; A61B 8/463; A61B 8/466; A61B 8/5207; A61B 8/5223; A61B 2560/0238; A61B 2560/0462; A61B 2562/0247; A61B 2562/0271; A61B 2562/028; A61B 2562/046

(57) ABSTRACT

A neurovascular ultrasonic imaging catheter is disclosed. The neurovascular ultrasonic catheter having a longitudinal axis, a proximal end, and a distal end, and the at least one ultrasound transducer array integrated within the distal end of the neurovascular ultrasonic catheter. The at least one ultrasound transducer array is configured for neuro intravascular ultrasound imaging of intracranial vessels and other intracranial structures, and one or more electrical interconnects configured for electrically coupling the neurovascular ultrasonic catheter with an imaging system to transmit signals corresponding to the neuro intravascular ultrasound imaging of the intracranial vessels to the imaging system. The at least one ultrasound transducer array is positioned side-facing or forward facing relative to the longitudinal axis of the catheter shaft and offset to the distal end of the neurovascular ultrasonic catheter.

### FIELD OF THE DISCLOSURE

The present disclosure relates generally to the field of ultrasonic imaging systems. More particularly, some embodiments relate to a neurovascular ultrasonic catheter, a neurovascular ultrasonic system for acquiring high-resolution intravascular images of intravascular vessels and structures, and a method or methods thereof.

### BACKGROUND OF THE DISCLOSURE

Ultrasound imaging has long been a cornerstone of medical diagnostics, offering non-invasive and real-time visualization of internal structures. In particular, assessing neurovascular structures within the brain is crucial for diagnosing and treating various conditions such as stroke, aneurysms, and vascular malformations. However, conventional imaging techniques often struggle to provide the resolution and detail needed for effective assessment of intracranial vessels due to the complexity and delicate nature of the vasculature. The main challenge with visualization is that other techniques must image through the skull, and its thick bone significantly degrades image quality.

Traditional methods for imaging neurovascular structures often include external ultrasound systems, which provide limited access and resolution for detailed visualization of intracranial blood vessels. Current intracranial imaging techniques, such as magnetic resonance imaging (MRI) or computed tomography (CT), while effective, can be limited by factors such as patient safety concerns (e.g., contrast use, radiation exposure), cost, and availability of equipment. Moreover, these systems are generally not suitable for direct, real-time imaging of intracranial vasculature, particularly in smaller or harder-to-reach regions.

As the field of medical imaging continues to evolve, there is a growing need for more advanced, high-resolution imaging solutions capable of directly visualizing the intracranial vasculature. The advent of micro-electromechanical systems (MEMS) technology in the field of ultrasound has opened new possibilities for developing miniaturized, high-frequency transducers that can be integrated into medical devices. MEMS-based ultrasound systems offer significant advantages over traditional/bulk piezoelectric ultrasound transducers, including smaller form factors, lower power consumption, and enhanced imaging resolution.

### SUMMARY OF THE DISCLOSURE

By way of introduction, the embodiments described below can include a neurovascular ultrasonic catheter. The neurovascular ultrasonic catheter comprises an ultrasonic catheter having a longitudinal axis, a proximal end, and a distal end. Further, the neurovascular ultrasonic catheter comprises at least one ultrasound transducer array integrated within a distal tip at the distal end of the neurovascular ultrasonic catheter. The at least one ultrasound transducer array may comprise one ultrasound transducer. Further, the at least one ultrasound transducer array is configured for neuro intravascular ultrasound imaging of intracranial vessels. Further, in one embodiment, the at least one ultrasound transducer array corresponds to a micro-electro-mechanical systems (MEMS) ultrasound transducer array. In another embodiment, the at least one ultrasound transducer array corresponds to a bulk PZT, piezocomposite, or single crystal piezoelectric, like a PMN-PT ultrasound transducer array. Further, in one embodiment, at least one catheter shaft is disposed between the distal and proximal ends of the neurovascular ultrasonic catheter. Further, one or more electrical interconnects are configured for electrically coupling the neurovascular ultrasonic catheter with an imaging system to transmit signals corresponding to the neuro intravascular ultrasound imaging of intracranial vessels to the imaging system. Further, in one embodiment, the at least one ultrasound transducer array is positioned side-facing relative to the longitudinal axis of the at least one catheter shaft and offset to the distal end of the neurovascular ultrasonic catheter. Further, in one embodiment, the at least one ultrasound transducer array is positioned forward-facing relative to the longitudinal axis of the at least one catheter shaft and offset to the distal end of the neurovascular ultrasonic catheter.

Further, a MEMS ultrasound transducer array comprises a piezoelectric micromachined ultrasonic transducer (PMUT) or a capacitive micromachined ultrasonic transducer (CMUT). Further, the PMUT ultrasound transducer array can be made from a piezocomposite PZT (Lead Zirconate Titanate) or epitaxial lead zirconate titanate (Epi-PZT) thin film. Further, in another embodiment, the transducer is made with bulk PZT, piezocomposite, or a single crystal piezoelectric such as Lead Magnesium Niobate-Lead Titanate (PMN-PT) or Lead Zinc Niobate-Lead Titanate (PZN-PT).

Further, the neurovascular ultrasonic catheter may further comprise a handle at the proximal end of the neurovascular ultrasonic catheter and be configured to control movement, orientation, and deflection of the neurovascular ultrasonic catheter. The handle further comprises an actuation mechanism configured to steer the catheter shaft to the desired angle.

In one embodiment, a neurovascular ultrasonic system is disclosed. The neurovascular ultrasonic system comprises a neurovascular ultrasonic catheter having a longitudinal axis, a proximal end, a distal end, and at least one ultrasound transducer array integrated within a distal tip at the distal end of the neurovascular ultrasonic catheter. Further, the at least one ultrasound transducer array is configured for neuro intravascular ultrasound imaging of intracranial vessels. The catheter shaft is disposed between the distal and proximal ends of the ultrasonic catheter and may comprise at least one additional lumen for guidewire insertion or therapeutic agent delivery. Further, the at least one ultrasound transducer array is positioned side-facing relative to the longitudinal axis of the catheter shaft and offset to the distal end of the neurovascular ultrasonic catheter. Further, an imaging system is communicatively coupled to the at least one ultrasound transducer array via one or more electrical interconnects, which may comprise flexible circuits and/or coaxial cables or any combination thereof. Further, the imaging system is configured to send signals to the at least one ultrasound transducer array and receive signals from the at least one ultrasound transducer array corresponding to the imaging of the intracranial vessels of a patient, perform one or more image processing techniques on the received signals to determine neurovascular structures, one or more medical abnormalities in the patient and one or more neurological diagnostic information, and generate one or more high resolution intravascular images of the intracranial vessels of the patient based on the one or more image processing techniques.

Further, the one or more image processing techniques comprise artificial intelligence-based advanced image reconstructions, adaptive filtering techniques to reduce noise and enhance signal-to-noise ratio, computational super resolution algorithms from multiple ultrasound frames to create one or more high-resolution intravascular images, and doppler processing to display blood flow information. Further, the one or more medical abnormalities include strokes, tumor localization and characterization, detection of cerebral aneurysms, arteriovenous malformations, abnormal blood vessels, neurodegenerative diseases, brain injury, and brain infections. Further, the one or more neurological diagnostic information includes the monitoring of intracranial pressure (ICP), guidance of brain biopsies, effects of medical treatments, guiding the delivery of the therapeutic agents, clot removal guidance, intracranial atherosclerosis disease (ICAD) assessment, aneurysm repair guidance, tumor ablation guidance, deep brain simulation, stem cell therapy guidance, monitoring effects of hypothermia therapy, and shunt placement guidance.

In one embodiment, a method for imaging neurovascular structures within a patient's brain using a neurovascular ultrasonic catheter is disclosed. Further, the method comprises steps of accessing the arterial or venous system of the brain through a radial or femoral access, then positioning the neurovascular ultrasonic catheter in the region of interest. Further, the neurovascular ultrasonic catheter having a longitudinal axis, a proximal end, a distal end, and the neurovascular ultrasonic catheter comprises at least one ultrasound transducer array integrated within a distal tip at the distal end of the neurovascular ultrasonic catheter. Further, the at least one ultrasound transducer array is configured for neuro intravascular ultrasound imaging of intracranial vessels, the catheter shaft is disposed between the distal and proximal ends of the ultrasonic catheter and may have at least one additional lumen for guidewire insertion or therapeutic agent delivery, and one or more electrical interconnects within the neurovascular ultrasonic catheter configured for electrically coupling the neurovascular ultrasonic catheter with an imaging system to transmit signals corresponding to the neuro intravascular ultrasound imaging of intracranial vessels. Further, the at least one ultrasound transducer array is positioned side-facing relative to the longitudinal axis of the catheter shaft and offset from the distal end of the neurovascular ultrasonic catheter; and utilizing the at least one ultrasound transducer array at the distal end of the neurovascular ultrasonic catheter to image stenosis, blood clots, wall thickness of aneurysms, tumors, and other malformations.

Other features and aspects of this disclosure will be apparent from the following description and the accompanying drawings.

### BRIEF DESCRIPTION OF THE DRAWINGS

The accompanying drawings illustrate various embodiments of systems, methods, and embodiments of various aspects of the disclosure. Any person of ordinary skill in the art will appreciate that the illustrated element boundaries (e.g., boxes, groups of boxes, or other shapes) in the figures represent one example of the various boundaries representative of the disclosed invention. It may be that in some examples, one element may be designed as multiple elements or that multiple elements may be designed as one element. In other examples, an element shown as an internal component of one element may be implemented as an external component in another and vice versa. Furthermore, elements may not be drawn to scale. Non-limiting and non-exhaustive descriptions of the present disclosure are described with reference to the following drawings. The components in the figures are not necessarily to scale, emphasis instead being placed upon the illustrated principles.

Various embodiments will hereinafter be described in accordance with the appended drawings, which are provided to illustrate and not to limit the scope of the disclosure in any manner, wherein similar designations denote similar elements, and in which:

FIG. 1 illustrates a schematic diagram of a side view of an embodiment of a neurovascular ultrasonic catheter that comprises at least one ultrasound transducer array and one or more electrical interconnects with a distal tip to assist with trackability in tortuous anatomy according to an embodiment of the present disclosure;

FIG. 2A illustrates a schematic diagram of a side view of an embodiment of a distal tip that comprises the at least one ultrasound transducer array and one or more electrical interconnects with a shortened overall length, where the at least one ultrasound transducer array is positioned side-facing according to an embodiment of the present disclosure;

FIG. 2B illustrates a schematic diagram of a side view of an embodiment of the distal tip with shortened overall length, which comprises of the at least one ultrasound transducer array and the one or more electrical interconnects with a shortened overall length where the at least one ultrasound transducer array is positioned side-facing according to an embodiment of the present disclosure;

FIG. 3 illustrates a schematic diagram of a side view of an embodiment of the distal tip, which comprises the at least one ultrasound transducer array and the one or more electrical interconnects where the at least one ultrasound transducer array is positioned forward-facing according to an embodiment of the present disclosure;

FIG. 4 illustrates an embodiment of a side-facing ultrasound transducer array for neurovascular ultrasonic imaging according to an embodiment of the present disclosure;

FIG. 5 illustrates an embodiment of a forward-facing ultrasound transducer array for neurovascular ultrasonic imaging according to an embodiment of the present disclosure;

FIG. 6 illustrates a schematic diagram showing a bi-directional deflection of at least one catheter shaft of the neurovascular ultrasonic catheter according to an embodiment of the present disclosure;

FIG. 7 illustrates a schematic diagram of a system flowchart showing communication between a neurovascular ultrasonic catheter, one or more electrical interconnects, and an imaging system to produce one or more high-resolution intravascular images of the intracranial vessels of the patient according to an embodiment of the present disclosure;

FIG. 8 illustrates a schematic diagram showing the neurovascular ultrasonic catheter accessing the arterial system through femoral access of a patient according to an embodiment of the present disclosure;

FIG. 9 illustrates a schematic diagram showing the neurovascular ultrasonic catheter accessing the arterial system through radial access of a patient according to an embodiment of the present disclosure;

FIG. 10 illustrates a schematic diagram showing one use case of the neurovascular ultrasonic catheter to characterize an aneurysm within neurovasculature of a patient according to an embodiment of the present disclosure;

FIG. 11A illustrates an embodiment of the neurovascular ultrasonic catheter where the at least one ultrasound transducer array is configured to be side-facing according to an embodiment of the present disclosure;

FIG. 11B Illustrates an embodiment of the neurovascular ultrasonic catheter where the at least one ultrasound transducer array is configured to be forward-facing according to an embodiment of the present disclosure; and

FIG. 12 illustrates a flowchart showing a method for imaging neurovascular structures within a patient's brain using the neurovascular ultrasonic catheter according to an embodiment of the present disclosure.

### DETAILED DESCRIPTION

The components of the embodiments as generally described and illustrated in the figures herein can be arranged and designed in a wide variety of different configurations. Thus, the following more detailed description of various embodiments, as represented in the figures, is not intended to limit the scope of the present disclosure but is merely representative of various embodiments. While various aspects of the embodiments are presented in drawings, the drawings are not necessarily drawn to scale unless specifically indicated.

Some embodiments of this disclosure, illustrating all its features, will now be discussed in detail. The words “comprising,” “having,” “containing,” and “including,” and other forms thereof, are intended to be equivalent in meaning and be open-ended in that an item or items following any one of these words is not meant to be an exhaustive listing of such item or items, or meant to be limited to only the listed item or items.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred systems and methods are now described. The terms “proximal” and “distal” are opposite directional terms. For example, the distal end of a device or component is the end of the component that is furthest from the practitioner during ordinary use. The proximal end refers to the opposite end, or the end nearest the practitioner during ordinary use.

Embodiments of the present disclosure will be described more fully hereinafter with reference to the accompanying drawings in which like numerals represent like elements throughout the several figures, and in which example embodiments are shown. Embodiments of the present disclosure may, however, be embodied in alternative forms and should not be construed as being limited to the embodiments set forth herein. The examples set forth herein are non-limiting examples and are merely examples among other possible examples.

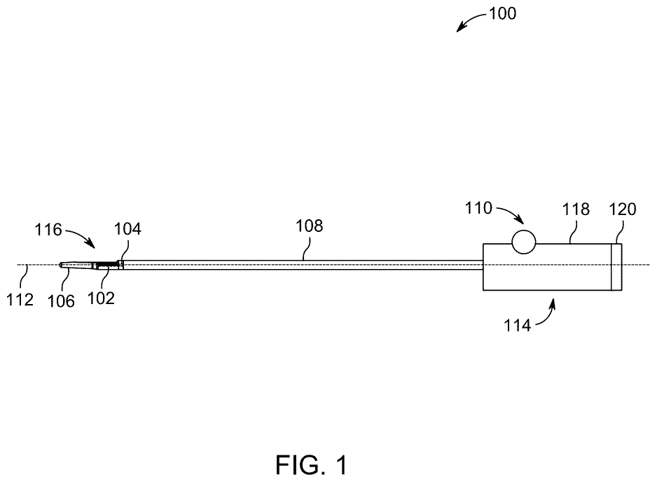

FIG. 1 illustrates a schematic diagram of a side view of an embodiment of a neurovascular ultrasonic catheter 100, which comprises at least one ultrasound transducer array 102, one or more electrical interconnects 104, and a distal tip 106 to assist with trackability in tortuous anatomy, according to an embodiment of the present disclosure.

In some embodiments, the neurovascular ultrasonic catheter 100 may comprise the at least one ultrasound transducer array 102, the one or more electrical interconnects 104, and at least one catheter shaft 108. In one embodiment, the at least one ultrasound transducer array 102 may comprise between 1 and 64 elements with an aperture measuring 0.5 mm to 1.5 mm in elevation and 0.5 mm to 6 mm in azimuth. In some embodiments, the neurovascular ultrasonic catheter 100 may further comprise an actuation mechanism 110. In some embodiments, the neurovascular ultrasonic catheter 100 may be utilized for diagnostic applications. In some embodiments, the neurovascular ultrasonic catheter 100 may be inserted into the brain vasculature or tissues surrounding the brain. Further, the neurovascular ultrasonic catheter 100 may be inserted via femoral, radial, or carotid access and guided into a patient's cerebral circulation. In one example embodiment, the neurovascular ultrasonic catheter 100 may be guided using an imaging technique (e.g., fluoroscopy or MRI).

In some embodiments, the neurovascular ultrasonic catheter 100 may be configured to produce detailed, real-time images of vasculature within the brain or surrounding tissues of a patient. In some embodiments, the neurovascular ultrasonic catheter 100 may be configured to use a plurality of ultrasonic waves to produce detailed, real-time images of vasculature within the brain or surrounding tissues of the patient. In some embodiments, the neurovascular ultrasonic catheter 100 may be configured to direct the plurality of ultrasonic waves at blood vessels inside the brain of the patient. Further, the plurality of ultrasonic waves may reflect off the tissues at different intensities, creating one or more dynamic images of neurovascular structures.

In some embodiments, the neurovascular ultrasonic catheter 100 may be configured to utilize the plurality of ultrasonic waves at different frequencies to perform one or more operations. Further, the one or more operations may comprise ultrasonic imaging. Further, the ultrasonic imaging may define a real-time visualization of vasculature.

In some embodiments, the neurovascular ultrasonic catheter 100 may have a longitudinal axis 112. Further, the neurovascular ultrasonic catheter 100 may comprise a distal end 116 and a proximal end 114. In some embodiments, the neurovascular ultrasonic catheter 100 may be constructed as an elongated, flexible structure defined along the longitudinal axis 112 that extends from the proximal end 114 to the distal end 116. In some embodiments, the proximal end 114 of the neurovascular ultrasonic catheter 100 may connect to external control and power systems (not shown). In some embodiments, the distal end 116 may interface directly with a neurovascular target site.

In some embodiments, the longitudinal axis 112 of the neurovascular ultrasonic catheter 100 may refer to a central line extending along the full length of the neurovascular ultrasonic catheter's body, from the proximal end 114 to the distal end 116. In some embodiments, the longitudinal axis 112 may serve as a reference line for defining and aligning one or more structural components and one or more functional components (e.g., sensors, transducers, fluid channels). In some embodiments, the longitudinal axis 112 of the neurovascular ultrasonic catheter 100 may facilitate maintaining orientation during navigation in tortuous neurovascular paths.

In some embodiments, the distal end 116 of the neurovascular ultrasonic catheter 100 may be referred to as an active region that interacts with a target anatomy. In some embodiments, the distal tip 106 of the neurovascular ultrasonic catheter 100 may be constructed with a tapered shape. Further, the distal tip 106 may be constructed with softer materials to prevent trauma to delicate vessels and easily navigate through the tortuous vasculature of the patient's brain. In some embodiments, the distal tip 106 of the neurovascular ultrasonic catheter 100 may comprise the at least one ultrasound transducer array 102. In some embodiments, the at least one ultrasound transducer array 102 may be integrated within the distal tip 106 at the distal end 116 of the neurovascular ultrasonic catheter 100. In some embodiments, the at least one ultrasound transducer array 102 may be configured for neuro intravascular ultrasound imaging of intracranial vessels.

In some embodiments, the at least one ultrasound transducer array 102 of the neurovascular ultrasonic catheter 100 may be configured to perform neuro intravascular ultrasound imaging of the intracranial vessels of the patient's brain. In some embodiments, the neurovascular ultrasonic catheter 100 may further comprise the one or more electrical interconnects 104. In some embodiments, the one or more electrical interconnects 104 may be integrated within the neurovascular ultrasonic catheter 100. Further, the one or more electrical interconnects 104 may be configured to electrically couple the neurovascular ultrasonic catheter 100 with an imaging system. Further, the one or more electrical interconnects 104 may be configured to enable the transmission of signals corresponding to the neuro intravascular ultrasound imaging of intracranial vessels from the at least one ultrasound transducer array 102 to the imaging system. It may be noted that a detailed description of the imaging system is explained in the following paragraphs.

In some embodiments, the distal end 116 of the neurovascular ultrasonic catheter 100 may be tapered at an angle (e.g., between 0° and 45°). In some embodiments, the distal tip 106 may be tapered at an angle and may be configured to facilitate ease of navigation through intravascular and intracranial vessels of a patient. Further, the distal tip 106 of the neurovascular ultrasonic catheter 100 may be made of materials with similar acoustic properties to those of blood. Further, the materials may include thermoplastic polyurethane (TPU), thermoplastic elastomer (TPE) (e.g. PEBA for example, available under the trade name PEBAX®), or a synthetic polymer (e.g. silicone).

In one embodiment, the distal tip 106 and catheter shaft 108 have an outer diameter ranging from approximately 0.5 mm to 3 mm, allowing access to small vessels while maintaining pushability and torque response.

In one example embodiment, the distal end 116 of the neurovascular ultrasonic catheter 100 may further comprise a plurality of radiopaque markers (not shown) and a plurality of sensors (not shown). In some embodiments, the plurality of radiopaque markers may be configured to provide fluoroscopic or angiographic image guidance to the neurovascular ultrasonic catheter 100 while advancing within the neurovascular structure of the patient's brain. In some embodiments, the plurality of radiopaque markers may be fabricated from biocompatible, high-contrast materials such as platinum-iridium alloy, gold, tantalum, or tungsten. Further, the plurality of radiopaque markers may be positioned at strategic intervals along the distal end 116 to provide clear positional feedback under imaging. In some embodiments, the plurality of sensors may comprise a pressure sensor, a flow rate sensor, a temperature sensor, etc.

In some embodiments, the pressure sensor may monitor intraluminal or perivascular pressure in real-time, allowing assessment of hemodynamic changes, vessel occlusion, or hemorrhagic risk. In some embodiments, the flow rate sensor may track cerebral blood flow velocity, assisting in evaluating the efficacy of clot dissolution or restoration of perfusion. In some embodiments, the temperature sensor may continuously record local tissue or fluid temperature near the target anatomy.

In some embodiments, the neurovascular ultrasonic catheter 100 may comprise at least one catheter shaft 108. In some embodiments, the catheter shaft 108 may be positioned between the distal end 116 and the proximal end 114 of the neurovascular ultrasonic catheter 100. In some embodiments, the catheter shaft 108 may extend along the longitudinal axis 112. In some embodiments, the catheter shaft 108 may serve as a primary structural and functional component of the neurovascular ultrasonic catheter 100. In some embodiments, the catheter shaft 108 may be configured to facilitate delivery of the ultrasonic energy, therapeutic agents, and sensor data (i.e., the intraluminal or perivascular pressure, cerebral blood flow velocity, and local tissue or fluid temperature near the target anatomy) along the longitudinal axis 112 of the neurovascular ultrasonic catheter 100.

In some embodiments, the at least one catheter shaft 108 may be configured to perform one or more operations. Further, the one or more operations may comprise at least transmission of electrical signals from the energy source to the at least one ultrasound transducer array 102 located at the distal end 116 of the neurovascular ultrasonic catheter 100 within the distal tip 106, fluid delivery from the fluid delivery system to the target anatomy, sensor wiring and data transmission, allowing real-time physiological monitoring.

In some embodiments, the catheter shaft 108 may extend along the longitudinal axis 112 of the neurovascular ultrasonic catheter 100. In some embodiments, the catheter shaft 108 may be configured to be reinforced with one or more elements (not shown). Further, the one or more elements may comprise a braided element, a coiled element, a polymeric element, a metallic element, etc. In some embodiments, one or more elements for reinforcing the catheter shaft 108 may be selected to enhance torque response, pushability, and kink resistance, especially in high-curvature regions of the vasculature of the patient's brain.

In some embodiments, the catheter shaft 108 may be constructed from biocompatible and flexible polymers such as thermoplastic polyurethane (TPU), thermoplastic elastomer (TPE) (e.g. PEBA for example, available under the trade name PEBAX®), or a synthetic polymer (e.g. silicone). Further, the biocompatible and flexible polymers for making the catheter shaft 108 may be selected to provide a balance between flexibility and mechanical strength to the catheter shaft 108. In some embodiments, the catheter shaft 108 may be configured to ensure a coordinated delivery of mechanical, electrical, fluidic, and sensory functions, enabling the neurovascular ultrasonic catheter 100 to perform targeted, minimally invasive interventions within the vasculature with high precision and control.

In some embodiments, the catheter shaft 108 may have at least one additional lumen for insertion of a guidewire or delivery of a therapeutic agent. In some embodiments, the at least one additional lumen may be constructed with optimal dimensions to accommodate a guidewire or to facilitate the delivery of therapeutic agents. In some embodiments, the at least one additional lumen may be centrally or eccentrically positioned within the catheter shaft 108. In some embodiments, the at least one additional lumen may be constructed with a low-friction PTFE (polytetrafluoroethylene) liner, polymeric additives, or polymeric coatings to reduce resistance during guidewire manipulation or fluid delivery.

In some embodiments, the proximal end 114 of the neurovascular ultrasonic catheter 100 may be located outside a patient's body during clinical use. Further, the proximal end 114 of the neurovascular ultrasonic catheter 100 may be connected to an energy source (i.e., an ultrasonic generator) (not shown), a catheter control unit (CCU) or handle (not shown), and an optional fluid delivery system (not shown). In some embodiments, the energy source may be configured to generate high frequency electrical signal having a frequency range of 1 MHz to 60 MHz). In some embodiments, the energy source may select the frequency for generating the high-frequency electrical signal based at least on a desired application (e.g., diagnostic imaging). In some embodiments, the energy source may comprise a frequency controller, power amplifier, waveform modulator, and impedance-matching circuitry to efficiently deliver energy to the neurovascular ultrasonic catheter 100.

In some embodiments, the CCU may correspond to an electronic system, a mechanical device, or a combination of both that may enable user(s) to operate the neurovascular ultrasonic catheter 100 manually, remotely, or through robotic assistance. In some embodiments, the CCU may provide a user interface to operate the neurovascular ultrasonic catheter 100. In some embodiments, the CCU may include a touchscreen display, control circuitry, data acquisition modules, and software platforms for controlling the movement of the neurovascular ultrasonic catheter 100. Further, the movement may define a bi-directional deflection or insertion and retraction (i.e., forward movement and backward movement), and shaft orientation (rotation) of the neurovascular ultrasonic catheter 100 while advancing within the neurovascular structure of the patient's brain. In one example embodiment, the CCU may comprise one or more wireless or wired communication interfaces for data logging, remote monitoring, or integration with external networks.

In some embodiments, the fluid delivery system may be configured to deliver therapeutic agents such as a thrombolytic agent (e.g., tPA), contrast media, saline for cooling, or microbubble contrast agents. In some embodiments, the fluid delivery system may further comprise a peristaltic or syringe infusion pump, fluid reservoirs, flow control valves, pressure sensors, and tubing connected to at least one additional lumen (not shown) within the neurovascular ultrasonic catheter 100. In some embodiments, the fluid delivery system may be operationally coupled with the CCU. Further, the CCU may be configured to allow an operator to control and adjust flow rate of the therapeutic agents. Further, the flow rate may range from 0.1 mL/min to 10 mL/min.

In some embodiments, the proximal end 114 of the neurovascular ultrasonic catheter 100 may further comprise a handle 118. In some embodiments, the handle 118 may be operationally coupled with the catheter shaft 108. In some embodiments, the handle 118 may be configured to control movement, orientation, and deflection of the neurovascular ultrasonic catheter 100. In some embodiments, the handle 118 may be configured to control movement, orientation, and deflection of the neurovascular ultrasonic catheter 100 during navigation through the intracranial vasculature. In one example embodiment, the handle 118 may comprise finger grips (not shown), thumbwheels (not shown), sliders (not shown), or rocker switches (not shown) for intuitive manipulation. In another example embodiment, the handle 118 may comprise a rotational collar (not shown). Further, the rotational collar may be configured to transmit torque or enable rotation of the distal tip 106 of the neurovascular ultrasonic catheter 100.

In some embodiments, the handle 118 may further comprise the actuation mechanism 110. Further, the actuation mechanism 110 of the handle 118 may be configured to steer the catheter shaft 108 at an angle. In some embodiments, the catheter shaft 108 of the neurovascular ultrasonic catheter 100 may be configured to be deflected bi-directionally in a range of angles from a neutral axis to 60 degrees in both positive and negative directions. In some embodiments, the catheter shaft 108 of the neurovascular ultrasonic catheter 100 may be configured to be deflected unidirectionally in a range of angles from a neutral axis to 60 degrees in one direction. In some other embodiments, the catheter shaft 108 of the neurovascular ultrasonic catheter 100 may be configured to be deflected in at least one direction in a range of angles from a neutral axis to 60 degrees in the at least one direction.

In some embodiments, the handle 118 may further comprise a receptacle 120. Further, the receptacle 120 may be communicatively coupled with the energy source. Further, the receptacle 120 may be configured to perform one or more operations. Further, the one or more operations may comprise at least reception of electrical signals from the energy source, reception of therapeutic agents from the fluid delivery system, sensor wiring, and data transmission, allowing real-time physiological monitoring.

In one embodiment, the neurovascular ultrasonic catheter 100 can comprise an electrically erasable programmable read-only memory (EEPROM) housed within the handle 118 or receptacle 120. The EEPROM is configured to store a range of data, including but not limited to, device identification data, sensor calibration data, and operational parameters. Additionally, for an embodiment where the neurovascular ultrasonic catheter 100 is single-use or disposable, the EEPROM is further configured with a usage tracking system, which could comprise a write-once counter, a timestamp upon connection to a system, or a lockout functionality once the neurovascular ultrasonic catheter 100 has been used.

In one embodiment, the neurovascular ultrasonic catheter 100 may comprise a temperature sensor, such as a thermocouple, at the distal end 116 of the neurovascular ultrasonic catheter 100, and embedded within the distal tip 106 or the body of the catheter shaft 108. The thermocouple can be configured to provide precise temperature readings where the neurovascular ultrasonic catheter 100 is in contact with the patient's blood or vascular structures to ensure the neurovascular ultrasonic catheter 100 does not reach temperatures that could cause damage to the anatomy. In one embodiment, the temperature sensor may be configured to detect an upper threshold for acceptable device temperature, and once the upper threshold is reached, send a signal to the imaging system to shut the neurovascular ultrasonic catheter 100.

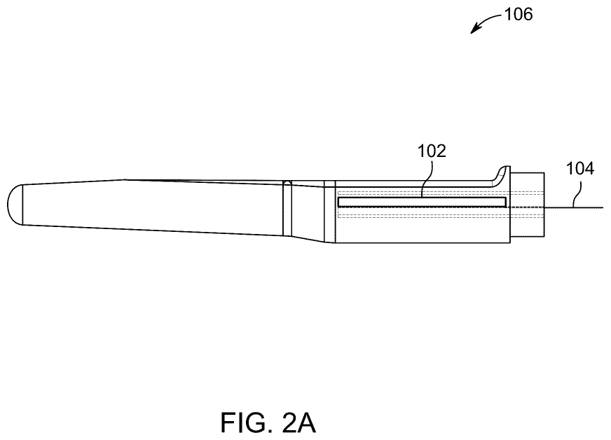

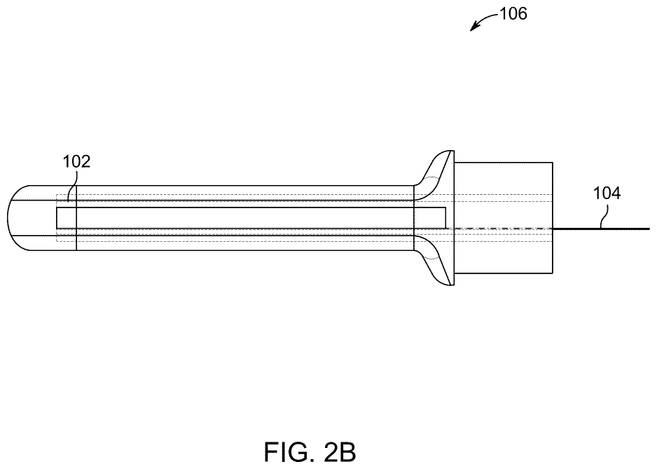

FIG. 2A illustrates a schematic diagram of a side view of an embodiment of the distal tip 106 which comprises the at least one ultrasound transducer array 102 and the one or more electrical interconnects 104 with a shortened overall length, where the at least one ultrasound transducer array 102 is positioned side-facing, according to an embodiment of the present disclosure. FIG. 2B illustrates a schematic diagram of a side view of an embodiment of the distal tip 106 with shortened overall length, which comprises the at least one ultrasound transducer array 102 and the one or more electrical interconnects 104 with a shortened overall length where the at least one ultrasound transducer array 102 is positioned side-facing, according to an embodiment of the present disclosure.

In some embodiments, the neurovascular ultrasonic catheter 100 may comprise the proximal end 114 and the distal end 116. In some embodiments, the proximal end 114 may enable coupling of the neurovascular ultrasonic catheter 100 with the external control and power systems. In some embodiments, the distal end 116 may interface directly with the neurovascular target site. In some embodiments, the distal end 116 of the neurovascular ultrasonic catheter 100 may further comprise the distal tip 106. In some embodiments, the distal tip 106 of the neurovascular ultrasonic catheter 100 may be exposed to the target anatomy.

In some embodiments, the distal tip 106 of the neurovascular ultrasonic catheter 100 may further comprise the at least one ultrasound transducer array 102. In some embodiments, the at least one ultrasound transducer array 102 may be configured for neuro intravascular ultrasound imaging of the intracranial vessels. In some embodiments, the at least one ultrasound transducer array 102 may include a capacitive micromachined ultrasonic transducer (CMUT) or a piezoelectric micromachined ultrasonic transducer (PMUT). Further, the at least one ultrasound transducer array 102 may be configured to operate at higher frequencies ranging from 1 MHz to 60 MHz, suitable for high-resolution imaging of small-caliber intracranial vessels.

In some embodiments, the PMUT may comprise a thin membrane and a piezoelectric film. Further, the PMUT may be configured to generate a plurality of ultrasound waves. Further, the generation of the plurality of ultrasound waves may rely on an interplay between the thin membrane's bending motion and the piezoelectric film's electromechanical response. Further, the thin membrane may be deposited with a piezoelectric material (such as AlN or PZT). Further, when an AC voltage is applied across the piezoelectric film, an inverse piezoelectric effect causes the piezoelectric film to expand or contract. Further, the thin membrane may be anchored at its edges and is designed to resonate at a specific frequency when excited.

Further, the piezoelectric film may be tightly bonded to the thin membrane. As the piezoelectric film expands or contracts due to the applied voltage, it induces bending in the thin membrane due to the mechanical strain mismatch. Further, the bending motion is amplified due to the thinness and compliance of the thin membrane, leading to significant out-of-plane displacement. As the thin membrane oscillates at ultrasonic frequencies, it creates pressure variations in the surrounding medium (e.g., air or water). Further, the pressure fluctuations propagate as ultrasonic waves. The resonance frequency of the thin membrane, along with its geometry and material properties, determines the frequency and efficiency of ultrasound generation. When an incident ultrasound wave strikes the thin membrane, it causes the thin membrane to oscillate. The mechanical deformation of the piezoelectric film generates a charge due to the direct piezoelectric effect, which may be measured as a voltage signal (i.e., the electrical signals). Moreover, the combination of the piezoelectric film and the thin membrane bending allows PMUTs to efficiently generate and receive ultrasonic waves, making them ideal for applications in medical imaging, fingerprint sensing, and non-destructive testing.

In some embodiments, the operation of the PMUT begins with the piezoelectric film, typically made from materials such as lead zirconate titanate (PZT) or aluminum nitride (AlN). When an alternating voltage is applied across the electrodes of the piezoelectric layer, the piezoelectric film undergoes in-plane strain due to the inverse piezoelectric effect. The strain in the piezoelectric film induces stress in the underlying membrane, leading to bending motion. Further, the thin membrane to which the piezoelectric film is attached is usually a thin silicon or composite structure. The bending of the thin membrane follows thin plate theory as described by the Kirchhoff-Love plate equations. Since PMUTs are clamped at the edges, the displacement profile of the thin membrane resembles that of a circular or rectangular diaphragm in a drum-like motion. For small displacements, the thin membrane undergoes linear bending, while at high driving voltages, nonlinear effects such as geometric stiffening come into play.

In some embodiments, the oscillatory motion of the thin membrane causes pressure variations in the surrounding medium (e.g., air or water), resulting in ultrasonic wave generation. The acoustic pressure p(r,t) at a distance r from the thin membrane may be modelled using Rayleigh's integral for a baffled piston. In one example embodiment, for airborne ultrasound, PMUTs are designed for resonance at 40-100 kHz, while in medical imaging, PMUTs operate in the MHz range, where the thin membrane motion efficiently couples into water or biological tissues.

In some embodiments, the at least one ultrasound transducer array 102 may be integrated within the distal tip 106 at the distal end 116 of the neurovascular ultrasonic catheter 100. In one example embodiment, the at least one ultrasound transducer array 102 may be mounted on the distal tip 106 of the distal end 116 of the neurovascular ultrasonic catheter 100. In another example embodiment, the at least one ultrasound transducer array 102 may be embedded within walls at the distal end 116 of the neurovascular ultrasonic catheter 100. In some embodiments, the at least one ultrasound transducer array 102 may be integrated within an atraumatic distal tip 106 to minimize the risk of trauma to delicate vascular walls. As illustrated in FIG. 2B, the ultrasound transducer 102 may be positioned in close proximity to the distal end 116 of the distal tip 106.

In some embodiments, the at least one ultrasound transducer array 102 may be configured for neuro intravascular ultrasound imaging, such as intracranial ultrasound or ultrasound localization microscopy (ULM). Further, the at least one ultrasound transducer array 102 may be configured to perform neuro intravascular ultrasound imaging by emitting high-frequency acoustic pulses (e.g., ranging from 1 MHz to 60 MHz). In some embodiments, the neuro intravascular ultrasound imaging may be tuned for detection of atherosclerotic plaque, thrombus formation, or vessel wall dissection, especially within intracranial arteries such as the middle cerebral artery, the anterior cerebral artery, the posterior communicating artery, the internal carotid artery, the ophthalmic artery, and the basilar artery.

In one embodiment, the at least one ultrasound transducer array 102 may be positioned side-facing relative to the longitudinal axis 112 of the catheter shaft 108 and offset to the distal end 116 of the neurovascular ultrasonic catheter 100. In some embodiments, the side-facing configuration of the at least one ultrasound transducer array 102 may be configured to enable a cross-sectional imaging of the intracranial vessels. In some embodiments, the side-facing configuration of the at least one ultrasound transducer array 102 may be suited for neuro intravascular ultrasound imaging and ultrasound elastography applications. In some embodiments, the at least one ultrasound transducer array 102 may be offset from the distal end of the distal tip 106 by a predefined threshold distance value (e.g., 0.25 mm to 15.0 mm) to safeguard the at least one ultrasound transducer array 102 during insertion and enable easier trackability through tortuous anatomy and enhance navigation accuracy in tortuous or occluded intracranial vessels.

As illustrated in FIG. 2B, in one embodiment, the at least one ultrasound transducer array 102 may be positioned near the distal end 116 of the distal tip 106. In some embodiments, the proximity of the at least one ultrasound transducer array 102 to the distal tip 106 may reduce the blind zone during insertion, allowing clinicians to more safely and confidently advance the neurovascular ultrasonic catheter 100 through complex neurovascular pathways.

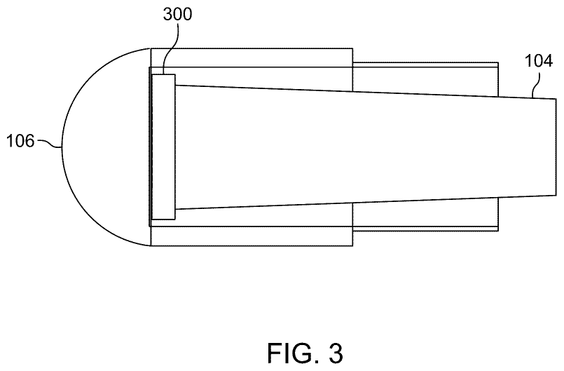

FIG. 3 illustrates a schematic diagram of a side view of an embodiment of the distal tip 106, which comprises of at least one ultrasound transducer array 300 and one or more electrical interconnects 104 where the at least one ultrasound transducer array 300 is positioned forward-facing according to an embodiment of the present disclosure. In some embodiments, the near-tip configuration of the at least one ultrasound transducer array 102 may facilitate real-time imaging of anatomical structures immediately beside the distal tip 106. In some embodiments, the near-tip positioning may be particularly advantageous for targeted therapeutic interventions, such as precise thrombus localization or lesion assessment.

In some embodiments, the distal tip 106 of the neurovascular ultrasonic catheter 100 may further comprise the at least one ultrasound transducer array 300. It may be noted that in one embodiment, the neurovascular ultrasonic catheter 100 may comprise the at least one ultrasound transducer array 102, and in another embodiment, the neurovascular ultrasonic catheter 100 may comprise the at least one ultrasound transducer array 300. In some embodiments, the at least one ultrasound transducer array 300 may be configured for neuro intravascular ultrasound imaging of the intracranial vessels. Further, the at least one ultrasound transducer array 300 may be configured to perform neuro intravascular ultrasound imaging by emitting high-frequency acoustic pulses (e.g., ranging from 1 MHz to 60 MHz). In some embodiments, the at least one ultrasound transducer array 300 may include a capacitive micromachined ultrasonic transducer (CMUT) or a piezoelectric micromachined ultrasonic transducer (PMUT). Further, the at least one ultrasound transducer array 300 may be configured to operate at higher frequencies ranging from 1 MHz to 60 MHz, suitable for high-resolution imaging of small-caliber intracranial vessels.

In another embodiment, the at least one ultrasound transducer array 300 may be positioned forward-facing relative to the longitudinal axis 112 of the catheter shaft 108 (i.e., oriented in a direction of the catheter's longitudinal axis 112 and aligned coaxially with the distal tip 106 of the neurovascular ultrasonic catheter 100). In some embodiments, the at least one ultrasound transducer array 300 may be positioned forward-facing relative to the longitudinal axis 112 of the catheter shaft 108 and positioned at the distal end 116 of the distal tip 106 of the neurovascular ultrasonic catheter 100. In some embodiments, the forward-facing ultrasound transducer array 300 may be configured to provide a longitudinal or axial imaging of the intracranial vessels. In some embodiments, the forward-facing ultrasound transducer array 300 may be essential for one or more operations where lateral visibility may be insufficient. In some embodiments, the one or more operations may comprise lesion characterization, guiding thrombolytic or thrombectomy devices, or navigating through occlusions and tight stenosis.

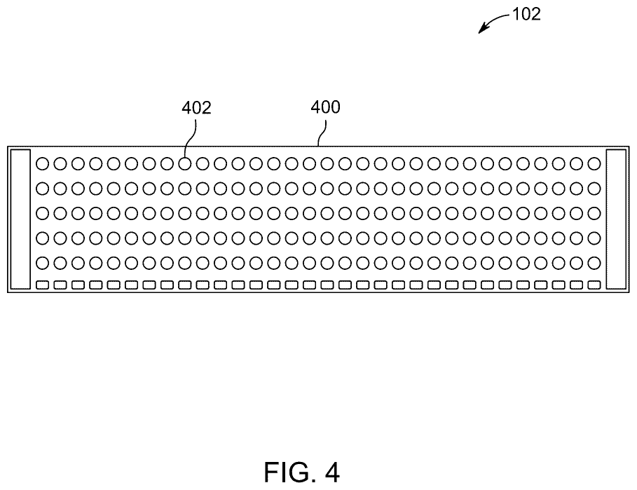

FIG. 4 illustrates an embodiment of a side-facing ultrasound transducer array 102 for neurovascular ultrasonic imaging, according to an embodiment of the present disclosure.

In some embodiments, the at least one ultrasound transducer array 102 may comprise a substrate 400. In one example embodiment, the substrate 400 of the ultrasonic transducer array 102 may be encapsulated with at least a biocompatible epoxy or polyimide coating. Further, the at least one ultrasound transducer array 102 may further comprise the one or more piezoelectric or capacitive elements 402. Further, the one or more piezoelectric or capacitive elements 402 may facilitate the at least one ultrasound transducer array 102 to perform neuro intravascular ultrasound imaging by emitting high-frequency acoustic pulses. Further, the one or more piezoelectric or capacitive elements 402 may be configured to receive the acoustic pulses. Further, the one or more piezoelectric or capacitive elements 402 may be configured to convert the received acoustic pulses into electrical signals. In some embodiments, the substrate 400 may be configured to provide electrical insulation and hemocompatibility to the transducer array 102. It may be noted that the transducer array 300 may also comprise the substrate 400, the one or more piezoelectric or capacitive elements 402.

Further, the one or more piezoelectric or capacitive elements 402 may facilitate the at least one ultrasound transducer array 102 to perform neuro intravascular ultrasound imaging by emitting high-frequency acoustic pulses (e.g., ranging from 1 MHz to 60 MHz). Further, the one or more piezoelectric or capacitive elements 402 may be configured to receive the acoustic pulses reflected from the walls of the intracranial vessels. Further, the one or more piezoelectric or capacitive elements 402 may be configured to convert the received acoustic pulses into electrical signals.

As illustrated in FIG. 2A and FIG. 2B the neurovascular ultrasonic catheter 100 may further comprise the one or more electrical interconnects 104. Further, the one or more electrical interconnects 104 may be disposed within the distal end 116 of the neurovascular ultrasonic catheter 100. In some embodiments, the one or more electrical interconnects 104 may be configured for electrically coupling the neurovascular ultrasonic catheter 100 with the imaging system. Further, the one or more electrical interconnects 104 may be configured to receive the electrical signals from the at least one ultrasound transducer array 102. Further, the one or more electrical interconnects 104 may be configured to transmit the electrical signals to the imaging system through the catheter shaft 108.

In some embodiments, the imaging system may be configured to receive the electrical signals corresponding to the neuro intravascular ultrasound imaging of the intracranial vessels. In some embodiments, the imaging system may comprise at least a processor or a controller. Further, the imaging system may comprise amplifiers, filters, and digital signal processors. Further, the imaging system may be configured to generate one or more high-resolution intravascular images of the intracranial vessels of the patient. Further, the imaging system may be configured to generate one or more high-resolution intravascular images of the intracranial vessels of the patient using doppler processing techniques. The imaging system may be configured to perform artificial intelligence-based advanced image reconstructions. Further, the imaging system may be configured to perform adaptive filtering techniques to reduce noise and enhance the signal-to-noise ratio. Further, the imaging system may be configured to perform computational super-resolution algorithms from multiple ultrasound frames to create a higher-resolution image. It may be noted that the imaging system is described in more detail in the following embodiments.

In some embodiments, the artificial intelligence-based advanced image reconstructions may improve image quality and diagnostic capability for the neurovascular ultrasound imaging in neurovascular procedures. Further, the AI-driven solutions may facilitate the ultrasound imaging system to address noise reduction, resolution enhancement, automated segmentation, and real-time interpretation of vascular structures like stenosis, thrombus formation, tortuous anatomy, intracranial atherosclerotic disease (ICAD), and aneurysms. In some embodiments, the neurovascular ultrasound catheter images often suffer from speckle noise (granular artifacts reducing contrast), limited spatial resolution (especially in small intracranial vessels), and motion artifacts due to blood flow and catheter movement.

In some embodiments, the imaging system may be configured to perform noise reduction and resolution enhancement using deep learning-based denoising (DNNs and GANs), convolutional neural networks (CNNs) trained on high-quality neurovascular ultrasound datasets to filter speckle noise while preserving vessel wall details, and generative adversarial networks (GANs) to generate high-resolution neurovascular ultrasound images from lower-quality acquisitions. In some embodiments, the imaging system may utilize one or more super-resolution AI techniques, such as super-resolution convolutional neural networks (SRCNN), to improve pixel density, allowing ultrasound images to rival high-resolution CT/MRI. In some embodiments, the artificial intelligence may facilitate the imaging system in reconstructing high-frequency details lost during catheter motion.

In some embodiments, the imaging system may be configured to perform adaptive beamforming with AI using AI-driven adaptive beamforming algorithms to adjust ultrasound signals in real time to improve penetration depth and contrast. Further, the imaging system may utilize machine learning models to optimize transducer signal processing to enhance vessel wall differentiation. For example, deep-learning models like U-Net may be used to remove speckle noise in neurovascular ultrasound, improving lumen clarity and plaque characterization.

In some embodiments, the imaging system may be configured to utilize artificial intelligence (AI) for real-time vascular structure detection & segmentation. In some embodiments, the AI may facilitate the imaging system to automate vessel wall detection, plaque/thrombus segmentation, and aneurysm flow assessment. In some embodiments, the imaging system may utilize deep learning for vessel wall detection. Further, the AI ensures consistent measurements of stenosis severity, plaque burden, and clot location. Further, the AI may be trained on thousands of neurovascular ultrasound cases to detect hypoechoic vs. hyperechoic thrombi, differentiating between recently formed vs. calcified clots.

In some embodiments, the AI may facilitate the imaging unit for real-time decision support & automated diagnosis. Further, the AI may analyze ultrasound catheter images in real-time, assisting interventional neuroradiologists with automated stenosis grading (e.g., 50%, 70%, 90% narrowing), risk prediction for aneurysm rupture (based on wall thickness, angulation, dome-to-neck ratio, wall stress, flow patterns, and thrombus load), and clot hardness estimation (predicts embolus response to thrombectomy devices).

In one example embodiment, Artificial Intelligence (AI) transforms interventional neuroradiology by enhancing stroke detection, aneurysm risk assessment, vessel segmentation, and intra-procedural guidance. AI-driven solutions integrate with modalities like neuro intravascular ultrasound imaging, digital subtraction angiography (DSA), CT angiography (CTA), and MRI to improve precision, reduce procedural time, and optimize patient outcomes.

In some embodiments, the AI reduces time-to-treatment for stroke, improves aneurysm detection, and enhances thrombectomy precision. In some embodiments, the AI improves neurovascular ultrasound image quality, vessel segmentation, and real-time catheter navigation. In some embodiments, the AI enables personalized treatment planning, optimizing outcomes for stroke and aneurysm patients. In some embodiments, the AI may drive future innovations in robotic neurovascular interventions and real-time multimodal imaging fusion.

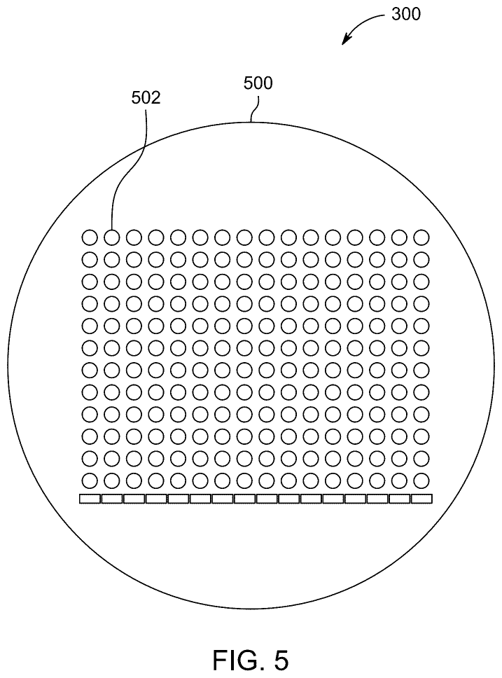

FIG. 5 illustrates an embodiment of a forward-facing ultrasound transducer array 300 for neurovascular ultrasonic imaging, according to an embodiment of the present disclosure.

In some embodiments, the at least one ultrasound transducer array 300 may comprise a substrate 500. In one example embodiment, the substrate 500 of the ultrasonic transducer array 300 may be encapsulated with at least a biocompatible material, such as epoxy or polyimide coating. Further, the at least one ultrasound transducer array 300 may further comprise one or more piezoelectric or capacitive elements 502. In some embodiments, the one or more piezoelectric or capacitive elements 502 may be oriented in a forward-facing configuration relative to the longitudinal axis 112 of the catheter shaft 108, enabling forward-looking neuro intravascular ultrasound imaging. The forward-facing arrangement may facilitate imaging of structures directly ahead of the distal tip 106 during neurovascular navigation. The one or more piezoelectric or capacitive elements 502 may be configured to emit high-frequency acoustic pulses and receive the reflected pulses from intracranial vascular structures, converting them into electrical signals.

Further, the one or more piezoelectric or capacitive elements 502 may facilitate the at least one ultrasound transducer array 300 to perform forward-looking neuro intravascular ultrasound imaging by emitting high-frequency acoustic pulses (e.g., ranging from 1 MHz to 60 MHz). In some embodiments, the forward-facing configuration allows for real-time visualization of occlusions, thrombi, and vessel bifurcations located directly in front of the distal tip 106 of the neurovascular ultrasonic catheter 100. The one or more piezoelectric or capacitive elements 502 may receive the acoustic signals reflected from the distal intracranial anatomy and convert the acoustic signals into electrical signals.

As illustrated in FIG. 2A and FIG. 2B the neurovascular ultrasonic catheter 100 may further comprise the one or more electrical interconnects 104. In some embodiments, the one or more electrical interconnects 104 may be configured for electrically coupling the neurovascular ultrasonic catheter 100 with the imaging system. Further, the one or more electrical interconnects 104 may be configured to receive the electrical signals from the at least one ultrasound transducer array 300. Further, the one or more electrical interconnects 104 may be configured to transmit and receive the electrical signals from the at least one ultrasound transducer array to and from the imaging system through the catheter shaft 108.

In some embodiments, the imaging system may be configured to receive the electrical signals corresponding to the neuro intravascular ultrasound imaging of the intracranial vessels. Further, the imaging system may comprise amplifiers, filters, and digital signal processors. Further, the imaging system may be configured to generate one or more high-resolution intravascular images of the intracranial vessels of the patient. Further, the imaging system may be configured to generate one or more high-resolution intravascular images of the intracranial vessels of the patient using doppler processing techniques. The imaging system may be configured to perform artificial intelligence-based advanced image reconstructions. Further, the imaging system may be configured to perform adaptive filtering techniques to reduce noise and enhance the signal-to-noise ratio. Further, the imaging system may be configured to perform computational super-resolution algorithms from multiple ultrasound frames to create a higher-resolution image. It may be noted that the imaging system is described in more detail in the following embodiments.

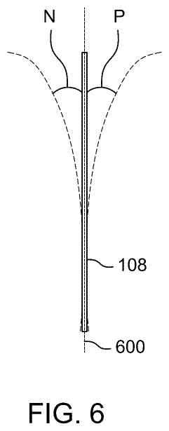

FIG. 6 illustrates a schematic diagram showing a bi-directional deflection of the catheter shaft 108 of the neurovascular ultrasonic catheter 100, according to an embodiment of the present disclosure.

In some embodiments, the catheter shaft 108 may extend along the longitudinal axis 112 of the neurovascular ultrasonic catheter 100. In some embodiments, the catheter shaft 108 may be made of flexible and biocompatible materials. In some embodiments, the neurovascular ultrasonic catheter 100 may be guided while advancing through the complex vasculature of the patient's brain. As illustrated in FIG. 6, the catheter shaft 108 may be configured to be deflected bi-directionally in a range of angles from the neutral axis 600 to 60 degrees in both positive and negative directions. In one example embodiment, the catheter shaft 108 may be configured to be deflected in a first angle P from the neutral axis 600 in the positive direction. Further, the first angle may define a range of angles between 0 degrees and 60 degrees. In one example embodiment, the catheter shaft 108 may be configured to be deflected in a second predefined angle N from the neutral axis 600 in the negative direction. Further, the second predefined angle may define a range of angles between 0 and 60 degrees.

In some embodiments, the proximal end 114 of the neurovascular ultrasonic catheter 100 may further comprise the handle 118. In some embodiments, the handle 118 may be operationally coupled with the catheter shaft 108. In some embodiments, the handle 118 may be configured to control movement, orientation, and deflection of the neurovascular ultrasonic catheter 100. In some embodiments, the handle 118 may be configured to control movement, orientation, and deflection of the neurovascular ultrasonic catheter 100 during navigation through the intracranial vasculature.

In some embodiments, the handle 118 may further comprise the actuation mechanism 110. Further, the actuation mechanism 110 of the handle 118 may be configured to deflect the catheter shaft 108 at an angle. Further, the catheter shaft 108 may be configured to be deflected bi-directionally to travel inside the complex vasculature of the patient's brain and/or to adjust the ultrasound transducer position to bring the desired target anatomy into view. Further, the one or more electrical interconnects 104 of the neurovascular ultrasonic catheter 100 may be configured to transmit the electrical signals to the imaging system through the catheter shaft 108.

In some embodiments, the imaging system may be electrically coupled with the neurovascular ultrasonic catheter 100 through the one or more electrical interconnects 104. In some embodiments, the imaging system may be configured to receive the electrical signals corresponding to the neuro intravascular ultrasound imaging of the intracranial vessels. In some embodiments, the imaging system may comprise at least a processor or a controller. In some embodiments, the imaging system may be configured to perform one or more image processing techniques on the received signals to determine neurovascular structures, one or more medical abnormalities in the patient, and one or more neurological diagnostic information. Further, the imaging system may be configured to generate one or more high-resolution intravascular images of the intracranial vessels of the patient based on one or more image processing techniques. In some embodiments, the neurovascular ultrasonic catheter 100 may be coupled with a display console. Further, the display console may be configured to display the one or more high-resolution intravascular images of the intracranial vessels of the patient.

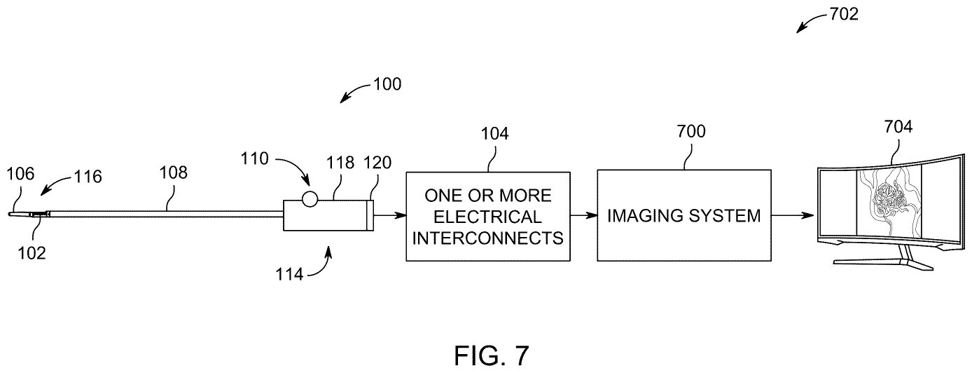

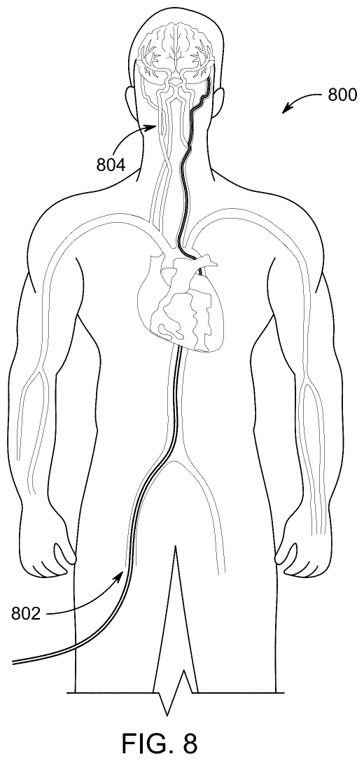

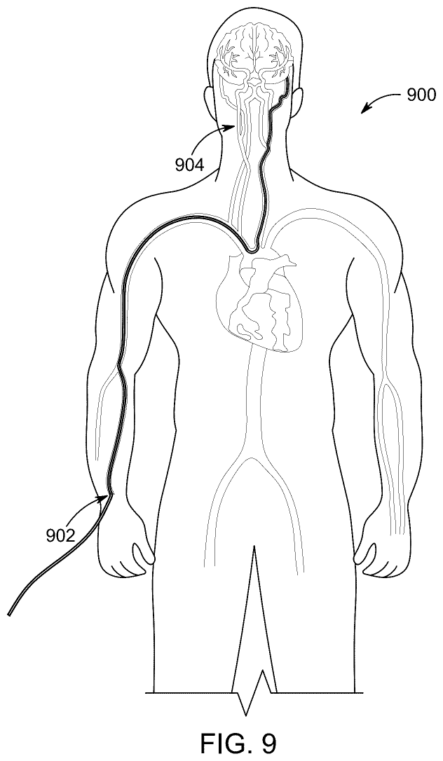

FIG. 7 illustrates a schematic diagram of a system flowchart showing communication between the neurovascular ultrasonic catheter 100, the one or more electrical interconnects 104, and an imaging system 700 to produce one or more high-resolution intravascular images of the intracranial vessels of the patient, according to an embodiment of the present disclosure. FIG. 8 illustrates a schematic diagram showing the neurovascular ultrasonic catheter 100 accessing the arterial system of a patient 800 through femoral access 802, according to an embodiment of the present disclosure. FIG. 9 illustrates a schematic diagram showing the neurovascular ultrasonic catheter 100 accessing the arterial system of a patient 900 through a radial access 902, according to an embodiment of the present disclosure.

In some embodiments, a neurovascular ultrasonic system 702 may comprise the neurovascular ultrasonic catheter 100, the at least one ultrasound transducer array 102, and the at least one catheter shaft 108. In some embodiments, the neurovascular ultrasonic system 702 may further comprise the one or more electrical interconnects 104, the imaging system 700, and a display console 704. In some embodiments, the neurovascular ultrasonic system 702 may be configured to perform neuro intravascular ultrasound imaging of intracranial vessels. Further, the neurovascular ultrasonic system 702 may be designed to support high-resolution, real-time imaging of neurovascular structures of the patient's brain. Further, the neurovascular ultrasonic system 702 may facilitate diagnostic and interventional procedures such as thrombectomy, aneurysm coiling, or stent placement.

In some embodiments, the neurovascular ultrasonic system 702 may comprise the neurovascular ultrasonic catheter 100. In some embodiments, the neurovascular ultrasonic catheter 100 may be utilized for diagnostic and therapeutic applications. In some embodiments, the neurovascular ultrasonic catheter 100 may be configured to produce detailed, real-time images of vasculature within the brain or surrounding tissues of the patient. In some embodiments, the neurovascular ultrasonic catheter 100 may be configured to use the plurality of ultrasonic waves to produce detailed, real-time images of vasculature within the brain or surrounding tissues of the patient. In some embodiments, the neurovascular ultrasonic catheter 100 may be configured to direct the plurality of ultrasonic waves at blood vessels inside a patient's brain. Further, the plurality of ultrasonic waves may reflect off the tissues at different intensities, creating one or more dynamic images of neurovascular structures.

In some embodiments, the neurovascular ultrasonic catheter 100 may be inserted into the brain vasculature or tissues surrounding the patient's brain. Further, the neurovascular ultrasonic catheter 100 may be inserted via the femoral access 802 or the radial access 902. In some embodiments, the neurovascular ultrasonic catheter 100 may be inserted through the femoral access 802 or radial access 902, depending on clinical requirements, physician preferences, and patient-specific anatomical considerations. Further, the neurovascular ultrasonic catheter 100 may be guided into the patient's cerebral circulation.

As illustrated in FIG. 8, the neurovascular ultrasonic catheter 100 may be configured to perform neuro intravascular ultrasound imaging of neurovasculature 804 of the patient 800. Further, the neurovascular ultrasonic catheter 100 may be inserted via the femoral access 802. In some embodiments, the neurovascular ultrasonic catheter 100 may be inserted into a common femoral artery or vein in the groin region of the patient 800. Following successful insertion, the neurovascular ultrasonic catheter 100 may be guided through the external iliac artery, common iliac artery, and into the abdominal and thoracic aorta, subsequently advancing through the aortic arch and into the internal carotid artery or vertebral artery, depending on the targeted intracranial territory. Further, the neurovascular ultrasonic catheter 100 may be guided under fluoroscopy or other imaging modalities.

As illustrated in FIG. 9, the neurovascular ultrasonic catheter 100 may be configured to perform neuro intravascular ultrasound imaging of neurovasculature 904 of the patient 900. Further, the neurovascular ultrasonic catheter 100 may be inserted via the radial access 902. Further, the radial access 902 involves accessing the radial artery or vein in the wrist of the patient 900. After insertion, the neurovascular ultrasonic catheter 100 may be advanced proximally through the brachial artery, the subclavian artery, and into the aortic arch, followed by selective catheterization of the cerebral arteries. In one example embodiment, the neurovascular ultrasonic catheter 100 may be guided using an imaging technique (e.g., fluoroscopy or MRI). In some embodiments, the neurovascular ultrasonic catheter 100 may be configured to utilize the plurality of ultrasonic waves at different frequencies to perform one or more operations. Further, the one or more operations may comprise ultrasonic imaging. Further, the ultrasonic imaging may define a real-time visualization of vasculature.

In some embodiments, the at least one ultrasound transducer array 102 of the neurovascular ultrasonic catheter 100 may be configured to perform neuro intravascular ultrasound imaging of the intracranial vessels of the patient's brain. Further, the at least one ultrasound transducer array 102 of the neurovascular ultrasonic catheter 100 may be configured to generate signals corresponding to the imaging of the intracranial vessels of the patient. In some embodiments, the neurovascular ultrasonic catheter 100 may further comprise the one or more electrical interconnects 104. In some embodiments, the one or more electrical interconnects 104 may comprise flexible circuits and/or coaxial cables or any combination thereof.

In some embodiments, the neurovascular ultrasonic system 702 may further comprise the imaging system 700. In some embodiments, the imaging system 700 may be communicatively coupled to the at least one ultrasound transducer array 102 via one or more electrical interconnects 104. Further, one or more electrical interconnects 104 may be configured for electrically coupling the neurovascular ultrasonic catheter 100 with the imaging system 700. Further, the one or more electrical interconnects 104 may be configured to enable the transmission of electrical signals corresponding to the neuro intravascular ultrasound imaging of intracranial vessels from the at least one ultrasound transducer array 102 to the imaging system 700.

In one example embodiment, the neurovascular ultrasonic system 702 may further comprise one or more electrical interconnects 104. Further, the one or more electrical interconnects 104 may be configured to electrically couple the neurovascular ultrasonic catheter 100 with the imaging system 700. Further, the one or more electrical interconnects 104 may be configured to enable the transmission of electrical signals corresponding to the neuro intravascular ultrasound imaging of intracranial vessels from the at least one ultrasound transducer array 102 to the imaging system 700.

In some embodiments, the imaging system 700 may be configured to send and receive the signals from the at least one ultrasound transducer array 102 corresponding to the imaging of the intracranial vessels of the patient. In some embodiments, the imaging system 700 may be configured to perform one or more image processing techniques on the received signals to determine neurovascular structures, one or more medical abnormalities in the patient, and one or more neurological diagnostic information. Further, the one or more image processing techniques may comprise artificial intelligence-based advanced image reconstructions, adaptive filtering techniques to reduce noise and enhance signal-to-noise ratio, computational super-resolution algorithms from multiple ultrasound frames to create the one or more high-resolution intravascular images, and doppler processing to display blood flow information. Further, the one or more medical abnormalities may include at least strokes, tumor localization and characterization, detection of cerebral aneurysms, arteriovenous malformations, intracranial atherosclerotic disease, abnormal blood vessels, neurodegenerative diseases, brain injury, and brain infections. Further, the one or more neurological diagnostic information may include at least monitoring intracranial pressure (ICP), guidance of brain biopsies, effects of medical treatments, guiding the delivery of the therapeutic agent, guidance of clot removal, aneurysm repair guidance, tumor ablation guidance, deep brain simulation, guidance of stem cell therapies, monitoring effects of hypothermia therapy, and guidance or placement of shunts.

In some embodiments, the neurovascular ultrasonic system 702 may further comprise the display console 704. Further, the display console 704 may be communicatively coupled with the imaging system 700. Further, the one or more generated high-resolution intravascular images of the intracranial vessels of the patient may be displayed on the display console 704, as illustrated in FIG. 7. Further, the intravascular images may include cross-sectional and/or longitudinal views of vessel walls, lumen, blood flow information, and/or 3D reconstructions of neurovascular structures.

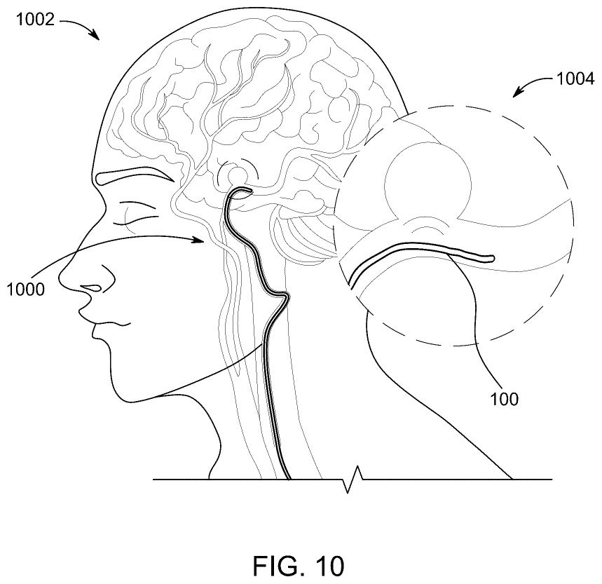

FIG. 10 illustrates a schematic diagram showing one use case of the neurovascular ultrasonic catheter 100 to characterize an aneurysm within neurovasculature 1000 of a patient 1002, according to an embodiment of the present disclosure.

In some embodiments, the neurovascular ultrasonic system 702 may be configured to detect a suspected vascular abnormality 1004, such as a cerebral aneurysm or arteriovenous malformation (AVM) located in the posterior intracranial circulation of the patient 1002, as illustrated in FIG. 10. In some embodiments, the neurovascular ultrasonic catheter 100 may be introduced through the femoral access 802 or radial access 902. Further, the neurovascular ultrasonic catheter 100 may be navigated under image guidance through the aortic arch and vertebral arteries, and advanced toward the basilar artery and posterior cerebral arteries.

In some embodiments, the distal tip 106 of the neurovascular ultrasonic catheter 100 may be integrated with the at least one ultrasound transducer array 102. In some embodiments, the at least one ultrasound transducer array 102 may be configured to transmit pulsed ultrasonic signals into the intracranial vessels while advancing into the cerebral arteries. Further, the at least one ultrasound transducer array 102 may be configured to receive ultrasonic signals reflected from the walls of the intracranial vessels.

Further, the at least one ultrasound transducer array 102 may be configured to generate electrical signals corresponding to the imaging of the intracranial vessels of the patient 1002. Further, the at least one ultrasound transducer array 102 may be configured to transmit electrical signals to the imaging system 700. Further, the imaging system 700 may be communicatively coupled to the at least one ultrasound transducer array 102 via the one or more electrical interconnects 104. Further, the imaging system 700 may be configured to send signals to the ultrasonic transducer array 102. Further, the imaging system 700 may be configured to receive signals from the at least one ultrasound transducer array 102 corresponding to the imaging of the intracranial vessels of the patient 1002. Further, the imaging system 700 may be configured to perform one or more image processing techniques on the received signals to detect the suspected vascular abnormality 1004. Further, the imaging system 700 may be configured to generate one or more high-resolution intravascular images of the intracranial vessels of the patient 1002 based on the one or more image processing techniques.

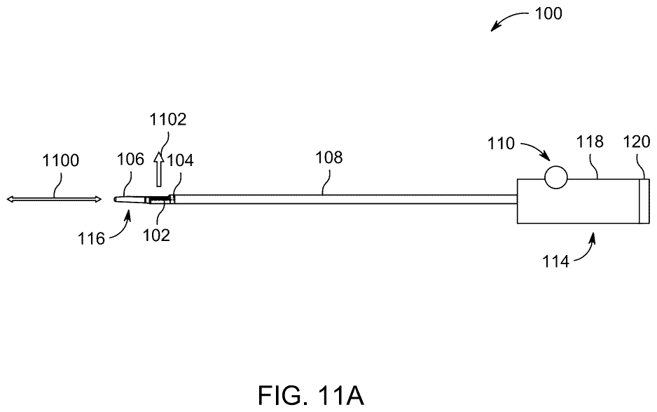

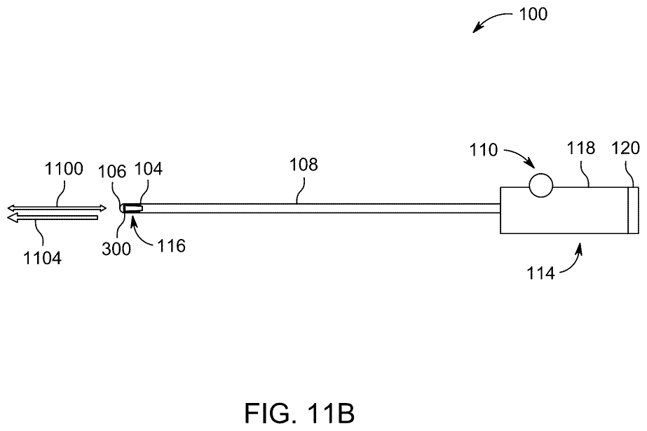

FIG. 11A illustrates an embodiment of the neurovascular ultrasonic catheter 100 where the at least one ultrasound transducer array 102 is configured to be side-facing, according to an embodiment of the present disclosure. FIG. 11B Illustrates an embodiment of the neurovascular ultrasonic catheter 100 where the at least one ultrasound transducer array 102 is configured to be forward-facing, according to an embodiment of the present disclosure.

In some embodiments, the neurovascular ultrasonic catheter 100 may comprise the longitudinal axis 112, the proximal end 114, and the distal end 116. In some embodiments, the neurovascular ultrasonic catheter 100 may be constructed as an elongated, flexible structure defined along the longitudinal axis 112 that extends from the proximal end 114 to the distal end 116. In some embodiments, the proximal end 114 of the neurovascular ultrasonic catheter 100 may connect to external control and power systems. In some embodiments, the distal end 116 may interface directly with a neurovascular target site. In some embodiments, the proximal end 114 of the neurovascular ultrasonic catheter 100 may be located outside a patient's body during clinical use. Further, the proximal end 114 of the neurovascular ultrasonic catheter 100 may be connected to the energy source (i.e., an ultrasonic generator), the catheter control unit (CCU), and the optional fluid delivery system.

In some embodiments, the distal end 116 of the neurovascular ultrasonic catheter 100 may be referred to as an active region that interacts with the target anatomy. In some embodiments, the distal end 116 of the neurovascular ultrasonic catheter 100 may be constructed with a tapered shape. Further, the distal end 116 may be constructed with a softer material or materials (i.e., lower durometer) to prevent trauma to delicate vessels. In some embodiments, the distal end 116 of the neurovascular ultrasonic catheter 100 may be tapered at an angle to facilitate ease of navigation through the intravascular vessels and the intracranial vessels of the patient. Further, the distal tip 016 may be made of materials that have similar acoustic properties to those of blood. Further, the materials may include thermoplastic polyurethane (TPU), thermoplastic elastomer (TPE) (e.g., PEBA, for example, available under the trade name PEBAX®), or a synthetic polymer (e.g., silicone).

In some embodiments, the distal end 116 of the neurovascular ultrasonic catheter 100 may comprise the distal tip 106. In some embodiments, the distal end 116 of the neurovascular ultrasonic catheter 100 may comprise the at least one ultrasound transducer array 102. In some embodiments, the at least one ultrasound transducer array 102 may be integrated within the distal tip 106 of the distal end 116 of the neurovascular ultrasonic catheter 100. In some embodiments, the at least one ultrasound transducer array 102 may be configured for neuro intravascular ultrasound imaging of intracranial vessels. In some embodiments, the at least one ultrasound transducer array 102 may correspond to a micro-electro-mechanical systems (MEMS) ultrasound transducer array 102. In some embodiments, the at least one ultrasound transducer array 102 may be configured for phased array imaging or linear array imaging, or a combination of phased array imaging and linear array imaging of the intracranial vessels of the patient.

In one embodiment, the at least one ultrasound transducer array 102 may be configured for phased array imaging. In phased array imaging, each element of the at least one ultrasound transducer array 102 may be activated with slight time delays (phasing) to electronically steer and focus the ultrasound beam in different directions without mechanically moving the at least one ultrasound transducer array 102. Further, the phased array imaging may allow for dynamic beam steering and focusing, enabling visualization of structures at varying depths and orientations.

In another embodiment, the at least one ultrasound transducer array 102 may be configured for linear array imaging. In linear array imaging, the elements of the at least one ultrasound transducer array 102 may be fired sequentially in a fixed, parallel orientation to produce a rectangular field of view. Further, the linear array imaging may provide high-resolution, cross-sectional images of the vessel walls and intraluminal structures to evaluate plaque burden, vessel morphology, or thrombus characterization.

In another embodiment, the at least one ultrasound transducer array 102 may be configured to operate in a hybrid mode, combining both phased array imaging and linear array imaging. Further, the combination of phased array imaging and linear array imaging may allow the neurovascular ultrasonic system 702 to utilize the beam steering capabilities of phased arrays for broader field-of-view and multi-directional scanning.

In some embodiments, the at least one ultrasound transducer array 102 may comprise a piezoelectric micromachined ultrasonic transducer (PMUT) or a capacitive micromachined ultrasonic transducer (CMUT). Further, in another embodiment, the transducer is made with bulk PZT, Piezocomposite, or a single crystal piezoelectric such as Lead Magnesium Niobate-Lead Titanate (PMN-PT) or Lead Zinc Niobate-Late Titanate (PZN-PT). In some embodiments, the PMUT can be constructed with an epitaxial lead zirconate titanate (Epi-PZT) thin film. In some embodiments, the at least one ultrasound transducer array 102 may be made from piezocomposite or single-crystal piezoelectric. In one embodiment, the neurovascular ultrasonic catheter 100 may be guided through a larger catheter or over a guidewire to advance through the complex vasculature of the patient's brain.

Referring to FIG. 11A, the neurovascular ultrasonic catheter 100 may be configured to be inserted and retracted (i.e., forward direction and backward direction), as shown by an arrow 1100. In one embodiment, the at least one ultrasound transducer array 102 may be positioned side-facing relative to the longitudinal axis 112 of the catheter shaft 108 and offset to the distal end 116 of the neurovascular ultrasonic catheter 100. Further, the at least one ultrasound transducer array 102 may be configured to emit a plurality of ultrasound waves towards the walls of the intracranial vessels, as shown by an arrow 1102. In some embodiments, the side-facing configuration of the at least one ultrasound transducer array 102 may be configured to enable a cross-sectional imaging of the intracranial vessels.

Referring to FIG. 11B, the neurovascular ultrasonic catheter 100 may be configured to be inserted and retracted (i.e., forward direction and backward direction), as shown by the arrow 1100. In another embodiment, the at least one ultrasound transducer array 102 may be positioned forward-facing relative to the longitudinal axis 112 of the catheter shaft 108. Further, the at least one ultrasound transducer array 102 may be configured to emit a plurality of ultrasound waves towards a field of view (FOV) of the distal tip 106, as shown by an arrow 1104. In some embodiments, the forward-facing configuration of the at least one ultrasound transducer array 102 may be configured to provide a longitudinal or axial imaging of the intracranial vessels

In some embodiments, the neurovascular ultrasonic catheter 100 may comprise at least one catheter shaft 108. In some embodiments, the catheter shaft 108 may be disposed between the distal end 116 and the proximal end 114 of the neurovascular ultrasonic catheter 100. In some embodiments, the catheter shaft 108 may extend along the longitudinal axis 112. In some embodiments, the catheter shaft 108 may be configured to facilitate delivery of the ultrasonic energy, therapeutic agents, and sensor data along the longitudinal axis 112 of the neurovascular ultrasonic catheter 100.

In some embodiments, the at least one catheter shaft 108 may extend along the longitudinal axis 112 of the neurovascular ultrasonic catheter 100. In some embodiments, the at least one catheter shaft 108 may be made of flexible and biocompatible materials. Further, the materials may comprise thermoplastic polyurethane (TPU), thermoplastic elastomer (TPE) (e.g., PEBA, for example, available under the trade name PEBAX®), or a synthetic polymer (e.g., silicone). Further, the biocompatible and flexible polymers for making the catheter shaft 108 may be selected to provide a balance between flexibility and mechanical strength to the catheter shaft 108. In some embodiments, the catheter shaft 108 may be configured to be deflected bi-directionally in a range of angles from the neutral axis to 60 degrees in both positive and negative directions.

In some embodiments, the proximal end 114 of the neurovascular ultrasonic catheter 100 may further comprise the handle 118. In some embodiments, the handle 118 may be operationally coupled with the catheter shaft 108. In some embodiments, the handle 118 may be configured to control movement, orientation, and deflection of the neurovascular ultrasonic catheter 100 during navigation through the intracranial vasculature.

In some embodiments, the handle 118 may further comprise the actuation mechanism 110. Further, the actuation mechanism 110 of the handle 118 may be configured to deflect the catheter shaft 108 to an angle. In some embodiments, the catheter shaft 108 of the neurovascular ultrasonic catheter 100 may be configured to be deflected bi-directionally a range of angles from the neutral axis 600 to 60 degrees in both positive and negative directions.

In one example embodiment, the actuation mechanism 110 may comprise a pull-line actuation assembly. Further, the pull-line actuation assembly may comprise at least one wire or string (i.e., a metal wire (e.g., high-tensile stainless steel or nitinol, etc.) or plastic (e.g., urethane, nylon, etc.), or a synthetic material (e.g., aramid, zylon, technora, spider wire, Ultra-High Molecular Weight Polyethylene (UHMWPE), or carbon fiber etc.). Further, the wire may be anchored at the distal end 116 of the neurovascular ultrasonic catheter 100. Further, the pull line may be routed through the inner lumen of the catheter shaft 108 and anchored in the handle 118. Further, the pull line may be routed through the shaft wall as axial elements. Further, the line may be applied with a controlled tension to achieve deflection of the shaft, thereby deflecting bi-directionally at an angle. In another example embodiment, shape-memory alloy (SMA) actuation assembly or electromechanical micro-actuation assembly may be used to provide electronic deflection capabilities to the neurovascular ultrasonic catheter 100, allowing deflection in at least one direction (e.g., bidirectional deflection shown as positive and negative in FIG. 6) from a neutral position.

In some embodiments, the at least one ultrasound transducer array 102 of the neurovascular ultrasonic catheter 100 may be configured to perform neuro intravascular ultrasound imaging of the intracranial vessels of the patient's brain. Further, the at least one ultrasound transducer array 102 of the neurovascular ultrasonic catheter 100 may be configured to send and receive signals corresponding to the imaging of the intracranial vessels of the patient. In some embodiments, the neurovascular ultrasonic catheter 100 may further comprise the one or more electrical interconnects 104. In some embodiments, the one or more electrical interconnects 104 may comprise flexible circuits and/or coaxial cables or any combination thereof.

In some embodiments, the neurovascular ultrasonic system 702 may further comprise the imaging system 700. In some embodiments, the imaging system 700 may be communicatively coupled to the at least one ultrasound transducer array 102 via the one or more electrical interconnects 104. Further, the one or more electrical interconnects 104 may be configured for electrically coupling the neurovascular ultrasonic catheter 100 with the imaging system 700. Further, the one or more electrical interconnects 104 may be configured to enable transmission of the signals corresponding to the neuro intravascular ultrasound imaging of intracranial vessels from the at least one ultrasound transducer array 102 to the imaging system 700.

In some embodiments, the imaging system 700 may be configured to receive the signals from the at least one ultrasound transducer array 102 corresponding to the imaging of the intravascular vessels of the patient. In some embodiments, the imaging system 700 may be configured to perform one or more image processing techniques on the received signals to determine neurovascular structures, one or more medical abnormalities in the patient, and one or more neurological diagnostic information. Further, the one or more image processing techniques may comprise of artificial intelligence based advanced image reconstructions, adaptive filtering techniques to reduce noise and enhanced signal to noise ratio, computational super resolution algorithms from multiple ultrasound frames to create the one or more high resolution intravascular images, and doppler processing to display blood flow information. Further, the one or more medical abnormalities may include at least strokes, tumor localization and characterization, detection of cerebral aneurysms, arteriovenous malformations, intracranial atherosclerotic disease, abnormal blood vessels, neurodegenerative diseases, brain injury, and brain infections. Further, the one or more neurological diagnostic information may include at least monitoring intracranial pressure (ICP), guidance of brain biopsies, effects of medical treatments, guiding the delivery of the therapeutic agent, guidance of clot removal, aneurysm repair guidance, tumor ablation guidance, deep brain simulation, guidance of stem cell therapies, monitoring effects of hypothermia therapy, and guidance of placement of shunts.

In some embodiments, the neurovascular ultrasonic system 702 may further comprise the display console 704. Further, the display console 704 may be communicatively coupled with the imaging system 700. Further, the generated one or more high-resolution intravascular images of the intracranial vessels of the patient may be displayed on the display console 704, as illustrated in FIG. 7. Further, the intravascular images may include cross-sectional and/or longitudinal views of the vessel walls, lumen, blood flow information, and/or 3D reconstructions of neurovascular structures.

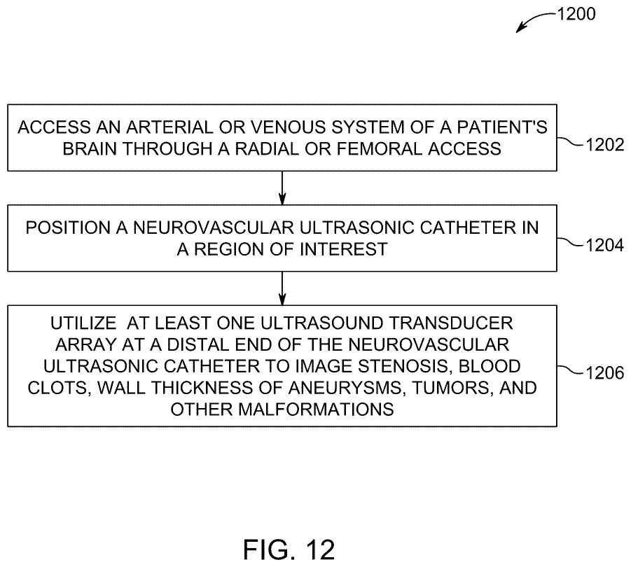

FIG. 12 illustrates a flowchart showing a method 1200 for imaging neurovascular structures within a patient's brain using the neurovascular ultrasonic catheter 100, according to an embodiment of the present disclosure.

At operation 1202, the arterial or venous system of the brain may be accessed through the radial access 902 or femoral access 802. In some embodiments, the insertion of the neurovascular ultrasonic catheter 100 through the femoral access 802 or radial access 902 may depend at least on clinical requirements, physician preferences, and patient-specific anatomical considerations. Further, the neurovascular ultrasonic catheter 100 may be guided into the cerebral circulation of the patient.

In one embodiment, the radial access 902 (e.g., distal radial or conventional radial) may be preferred in cases where a lower risk of bleeding and early ambulation is desired. Further, the radial access 902 may be used with smaller guide catheters (3 to 6 F). Further, the radial access 902 may be more challenging due to tortuous anatomy (i.e., subclavian and brachiocephalic routes). In another embodiment, the femoral access 802 (i.e., common femoral artery-CFA) may be traditionally a gold standard for neurovascular interventions. Further, the femoral access 802 may allow larger-caliber guide catheters (6 to 10 F) and may be preferred for complex interventions. Further, the femoral access 802 may provide a more direct approach to the aortic arch and cerebrovascular system. In another embodiment, a jugular or femoral venous access may be used for venous interventions, such as dural venous sinus stenting or thrombectomy in cases of cerebral venous sinus thrombosis (CVST).

In one embodiment, the radial access 902 may utilize at least one puncture technique, such as a modified standard Seldinger technique using ultrasound guidance to puncture the radial artery. Further, the radial access 902 may utilize a 3 to 6 F radial sheath. In another embodiment, the femoral access 802 may utilize the puncture technique, such as a standard Seldinger technique via fluoroscopic guidance, to access the CFA. Further, the femoral access 802 may utilize a 6 to 10 F sheath for more robust support.

In some embodiments, the neurovascular ultrasonic catheter 100 may be navigated into the aortic arch. Further, the neurovascular ultrasonic catheter 100 may be guided into the aortic arch and engaged into the origin of the target artery (e.g., the right internal carotid artery (ICA) from the brachiocephalic trunk or the left ICA directly from the aortic arch). Further, under fluoroscopic guidance, the neurovascular ultrasonic catheter 100 may be manipulated into the internal carotid artery (ICA) or vertebral artery (VA). Further, one or more guidewires may be used to navigate past tortuous anatomy. Further, once the target artery is engaged, an exchange maneuver may be performed. Further, the exchange maneuver may comprise one or more operations. At an operation, a stiff wire may be used to maintain vessel position. At another operation, an embodiment of the neurovascular ultrasonic catheter 100 with the at least one additional lumen may be advanced over the wire. Further, for complex cases, an intermediate catheter may be inserted for added stability.

In one example embodiment, a guidewire may be advanced to the area of interest. Further, for complex cases, an intermediate catheter may be inserted over the guidewire through the at least one additional lumen to provide added stability. Further, once the intermediate catheter is positioned, the guidewire may be removed.

Further, in embodiments of the neurovascular ultrasonic catheter 100 without the at least one additional lumen, a guidewire may first be advanced to the area of interest. Then, an intermediate catheter may be inserted over the guidewire until it is positioned in the area of interest. The guidewire may be removed and exchanged for the neurovascular ultrasonic catheter 100, which may be inserted into and advanced through the intermediate catheter to the area of interest.

At operation 1204, the neurovascular ultrasonic catheter 100 may be positioned in the region of interest. Further, the neurovascular ultrasonic catheter 100 may have the longitudinal axis 112, the proximal end 114, and the distal end 116. Further, the distal end 116 of the neurovascular ultrasonic catheter 100 may be tapered at an angle to facilitate ease of navigation through the intravascular vessels of the patient. Further, the distal tip 106 is made of materials which have acoustic properties matched to those of blood, the materials may comprise thermoplastic elastomer (TPE) (e.g., PEBA, for example, available under the trade name PEBAX®), or a synthetic polymer (e.g., silicone).

Further, the neurovascular ultrasonic catheter 100 may comprise of the at least one ultrasound transducer array 102, the catheter shaft 108, and one or more electrical interconnects 104. Further, the at least one ultrasound transducer array 102 may be integrated within the distal tip 106 at the distal end 116 of the neurovascular ultrasonic catheter 100. Further, the at least one ultrasound transducer array 102 may be configured for neuro intravascular ultrasound imaging of the intracranial vessels. Further, the at least one ultrasound transducer array 102 may correspond to a micro-electro-mechanical systems (MEMS) ultrasound transducer array 102.

Moreover, the catheter shaft 108 may be disposed between the distal end 116 and the proximal end 114 of the neurovascular ultrasonic catheter 100. Further, the catheter shaft 108 may comprise the at least one additional lumen for insertion of a guidewire or therapeutic agent delivery. Further, the catheter shaft 108 may be made of flexible and biocompatible materials. Further, the materials may comprise thermoplastic polyurethane (TPU), thermoplastic elastomer (TPE) (e.g., PEBA for example, available under the trade name PEBAX®), or a synthetic polymer (e.g., silicone). Further, the catheter shaft 108 may be configured to be deflected bi-directionally in a range of angles from the neutral axis to 60 degrees in both positive and negative directions. Furthermore, the one or more electrical interconnects 104 within the neurovascular ultrasonic catheter 100 may be configured for electrically coupling the neurovascular ultrasonic catheter 100 with the imaging system 700. Further, the one or more electrical interconnects 104 may be configured to transmit signals corresponding to the neuro intravascular ultrasound imaging of intracranial vessels from the at least one ultrasound transducer array 102 to the imaging system 700. Further, the one or more electrical interconnects 104 may comprise of flexible circuits and/or coaxial cables or any combination thereof. In some embodiments, the imaging system 700 may be electrically coupled with the neurovascular ultrasonic catheter 100 through the one or more electrical interconnects 104.

In some embodiments, the imaging system 700 may be configured to receive the electrical signals corresponding to the neuro intravascular ultrasound imaging of the intracranial vessels. Further, the imaging system 700 may be configured to generate one or more high-resolution intravascular images of the intracranial vessels of the patient based on one or more image processing techniques. In some embodiments, the neurovascular ultrasonic catheter 100 may be coupled with the display console 704. Further, the display console 704 may be configured to display one or more high-resolution intravascular images of the intracranial vessels of the patient.

In some embodiments, the neurovascular ultrasonic catheter 100 may be advanced over a guidewire to navigate into the target vessel. Further, a digital subtraction angiography (DSA) may be performed to confirm positioning. Further, a contrast injection through the neurovascular ultrasonic catheter 100 may provide a fluoroscopic roadmap. In some embodiments, a balloon guide catheter may be used for flow control. Further, the neurovascular ultrasonic catheter 100 may be used as an aspiration catheter or to deliver stent retrievers to remove clots.

In some embodiments, a plurality of tools and techniques may be used to guide catheter placement such as, a fluoroscopic roadmap & digital subtraction angiography (DSA) that is a live contrast-enhanced X-ray imaging to visualize vascular anatomy in real-time, a cone beam CT (CBCT) which provides 3D vascular reconstructions during procedures (used in embolization or AVM treatment), electromagnetic navigation that uses 3D electromagnetic tracking for precision catheter placement, and pressure and flow-guided techniques that uses fractional flow reserve (FFR) wires to measure hemodynamic significance in intracranial stenosis.

At operation 1206, the at least one ultrasound transducer array 102 at the distal end 116 of the neurovascular ultrasonic catheter 100 may be utilized to image stenosis, blood clots, wall thickness of aneurysms, tumors, and other malformations. In some embodiments, the at least one ultrasound transducer array 102 may be integrated within the distal tip 106 of the distal end 116 of the neurovascular ultrasonic catheter 100. Further, the at least one ultrasound transducer array 102 may correspond to a micro-electro-mechanical systems (MEMS) ultrasound transducer array 102. In some embodiments, the at least one ultrasound transducer array 102 of the neurovascular ultrasonic catheter 100 may be configured to perform neuro intravascular ultrasound imaging of the intracranial vessels of the patient's brain.

In some embodiments, the neurovascular ultrasonic catheter 100 may be utilized in one or more applications. Further, the one or more applications may comprise neuro intravascular ultrasound imaging, flow assessment, microvascular mapping, ultrasound-enhanced thrombolysis, plaque ablation, aneurysm management, etc. In some embodiments, the other applications may comprise real-time stroke detection, monitoring intracranial pressure (ICP), tumor localization and characterization, detection of cerebral aneurysms, arteriovenous malformation (AVM) imaging, early detection of neurodegenerative diseases, guidance for brain biopsies, monitoring brain injury, visualizing brain infections, monitoring the effects of medical treatments, targeted drug delivery, clot removal guidance, aneurysm repair guidance, tumor ablation guidance, deep brain stimulation (DBS) electrode placement, drainage of hematomas, opening of the blood brain barrier, guiding stem cell therapies, monitoring the effects of hypothermia therapy, and shunt placement guidance.

In some embodiments, the neuro intravascular ultrasound imaging may define a high-resolution visualization of intracranial vessel walls, plaque burden, stenosis, and aneurysms. Further, the flow assessment may define the evaluation of cerebral blood flow and velocity. Further, the microvascular mapping may define the visualization of microvasculature and collateral circulation. Furthermore, the ultrasound-enhanced thrombolysis may define the breakdown of clots in ischemic stroke by cavitation and mechanical vibration, often combined with thrombolytic drugs. Further, the plaque ablation may define disintegration of atherosclerotic plaques or calcifications using focused ultrasonic pulses. Thereafter, the aneurysm management may define assisting in endovascular coiling by clearing thrombus or visualizing aneurysmal necks.

The present invention introduces a novel neurovascular ultrasonic catheter 100 that incorporates MEMS ultrasound transducer technology. The neurovascular ultrasonic catheter 100 allows for high-resolution, neuro intravascular ultrasound imaging of intracranial vessels, providing a valuable tool for real-time assessment and diagnosis of neurovascular conditions. The neurovascular ultrasonic catheter 100 is designed to be flexible and navigable, enabling ease of use in the tortuous and narrow intracranial vasculature. Additionally, the integration of MEMS ultrasound transducers (i.e., the at least one ultrasound transducer array 102 or 300) enables the capture of high-frequency ultrasound images, offering greater spatial resolution and the ability to visualize smaller vascular structures that may be difficult to detect with traditional imaging techniques.

The disclosed invention offers a solution to the limitations of current imaging methods by providing a minimally invasive, high-resolution imaging tool specifically designed for intracranial ultrasound (ICU) procedures. The neurovascular ultrasonic system 702 features a compact, steerable catheter with integrated MEMS-based ultrasound transducers, offering improved imaging capabilities that can enhance the diagnosis and treatment of neurovascular diseases. Furthermore, the combination of advanced signal processing and imaging technology enables real-time, detailed imaging of the neurovascular structures, allowing clinicians to assess both the vessel anatomy and the flow characteristics of blood within the brain.

In summary, the present invention addresses the need for a sophisticated, minimally invasive imaging tool capable of providing high-resolution, real-time ultrasound imaging of the intracranial vasculature. By leveraging MEMS ultrasound transducer technology, this neurovascular ultrasonic catheter offers a significant advancement in the field of intracranial ultrasound imaging, with potential applications in both clinical diagnostics and therapeutic interventions.

While there is shown and described herein certain specific structures embodying various embodiments of the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed is:

1. A neurovascular ultrasonic catheter having a longitudinal axis, a proximal end, and a distal end, the neurovascular ultrasonic catheter comprising:
at least one ultrasound transducer array integrated at the distal end of the neurovascular ultrasonic catheter, wherein at least one ultrasound transducer array is configured for neuro intravascular ultrasound imaging of intracranial vessels and other intracranial structures;
at least one catheter shaft disposed between the distal end and the proximal end of the neurovascular ultrasonic catheter; and
one or more electrical interconnects configured for electrically coupling the neurovascular ultrasonic catheter with an imaging system to transmit signals corresponding to the neuro intravascular ultrasound imaging of the intracranial vessels to the imaging system,
wherein the at least one ultrasound transducer array is positioned side-facing relative to the longitudinal axis of the at least one catheter shaft and offset to the distal end of the neurovascular ultrasonic catheter.

2. The neurovascular ultrasonic catheter of claim 1, wherein the distal end of the neurovascular ultrasonic catheter is tapered at an angle to facilitate ease of navigation through the intracranial vessels of a patient.

3. The neurovascular ultrasonic catheter of claim 1, wherein the distal end is made of materials that have similar acoustic properties to those of blood, the materials include at least one thermoplastic polyurethane (TPU), thermoplastic elastomer (TPE) (e.g., PEBA for example, available under the trade name PEBAX®), or a synthetic polymer (e.g., silicone).

4. The neurovascular ultrasonic catheter of claim 1, wherein the at least one ultrasound transducer array is positioned forward-facing relative to the longitudinal axis of the at least one catheter shaft and positioned near the distal end of the neurovascular ultrasonic catheter.

5. The neurovascular ultrasonic catheter of claim 1, wherein the at least one ultrasound transducer array corresponds to a micro-electro-mechanical systems (MEMS) ultrasound transducer array.

6. The neurovascular ultrasonic catheter of claim 1, wherein the at least one ultrasound transducer array comprises a piezoelectric micromachined ultrasonic transducer (PMUT).

7. The neurovascular ultrasonic catheter of claim 1, wherein the at least one ultrasound transducer array comprises a piezoelectric micromachined ultrasonic transducer (PMUT) with an epitaxial lead zirconate titanate (Epi-PZT) thin film substrate.

8. The neurovascular ultrasonic catheter of claim 1, wherein the at least one ultrasound transducer array comprises at least a capacitive micromachined ultrasonic transducer (CMUT).

9. The neurovascular ultrasonic catheter of claim 1, wherein the at least one ultrasound transducer array comprises bulk PZT.

10. The neurovascular ultrasonic catheter of claim 1, wherein the at least one ultrasound transducer array comprises a piezocomposite.

11. The neurovascular ultrasonic catheter of claim 1, wherein the at least one ultrasound transducer array comprises a single-crystal piezoelectric.

12. The neurovascular ultrasonic catheter of claim 1, wherein the at least one ultrasound transducer array is configured for phased array imaging of the intracranial vessels of the patient.

13. The neurovascular ultrasonic catheter of claim 1, wherein the at least one ultrasound transducer array is configured for linear array imaging of the intracranial vessels of the patient.

14. The neurovascular ultrasonic catheter of claim 1, wherein the at least one ultrasound transducer array is configured for a combination of phased array and linear array imaging of the intracranial vessels of the patient.

15. The neurovascular ultrasonic catheter of claim 1, wherein the at least one catheter shaft is made of flexible and biocompatible materials, the materials comprise thermoplastic polyurethane (TPU), thermoplastic elastomer (TPE) (e.g., PEBA), polyamide polymer (e.g., nylon), or a synthetic polymer (e.g., silicone).

16. The neurovascular ultrasonic catheter of claim 1, wherein the at least one catheter shaft further comprises at least one additional lumen for insertion of a guidewire or therapeutic agent delivery.

17. The neurovascular ultrasonic catheter of claim 1, wherein the at least one catheter shaft is configured to be deflected in at least one direction from a neutral axis to 60 degrees.

18. The neurovascular ultrasonic catheter of claim 1, further comprising a handle at the proximal end of the neurovascular ultrasonic catheter and configured to control movement, orientation, and deflection of the neurovascular ultrasonic catheter, wherein the handle further comprises an actuation mechanism configured to deflect the at least one catheter shaft in a multitude of angles.

19. The neurovascular ultrasonic catheter of claim 1, wherein the one or more electrical interconnects comprise flexible circuits.

20. The neurovascular ultrasonic catheter of claim 1, wherein the one or more electrical interconnects comprise coaxial cables.

21. The neurovascular ultrasonic catheter of claim 1, wherein the one or more electrical interconnects comprise any combination of flexible circuits and coaxial cables.

22. The neurovascular ultrasonic catheter of claim 18, further comprises an electrically erasable programmable read-only memory (EEPROM) housed within the handle or receptacle and configured to store a range of data, including device identification data, sensor calibration data, and operational parameters.

23. The neurovascular ultrasonic catheter of claim 1, further comprises a temperature sensor, such as a thermocouple, at the distal end of the neurovascular ultrasonic catheter and is configured to provide precise temperature readings where the neurovascular ultrasonic catheter is contacting the patient blood or vascular structures to ensure the neurovascular ultrasonic catheter does not reach temperatures which can damage to anatomy.

24. The neurovascular ultrasonic catheter of claim 1, further comprises a pressure sensor at the distal end of the neurovascular ultrasonic catheter and is configured to provide intraluminal or perivascular pressure in real-time, allowing assessment of hemodynamic changes, vessel occlusion, or haemorrhagic risk.

25. The neurovascular ultrasonic catheter of claim 1, further comprises a flow rate sensor at the distal end of the neurovascular ultrasonic catheter and is configured to track cerebral blood flow velocity, assisting in evaluating the efficacy of clot dissolution or restoration of perfusion.

26. A neurovascular ultrasonic system comprising:
a neurovascular ultrasonic catheter having a longitudinal axis, a proximal end, and a distal end;
at least one ultrasound transducer array integrated at the distal end of the neurovascular ultrasonic catheter, wherein at least one ultrasound transducer array is configured for neuro intravascular ultrasound imaging of intracranial vessels and other intracranial structures;
at least one catheter shaft disposed between the distal end and the proximal end of the neurovascular ultrasonic catheter,
wherein the at least one ultrasound transducer array is positioned side-facing relative to the longitudinal axis of the at least one catheter shaft and offset to the distal end of the neurovascular ultrasonic catheter; and
an imaging system communicatively coupled to the at least one ultrasound transducer array via one or more electrical interconnects, wherein the imaging system is configured to:
transmit signals to the at least one ultrasound transducer array;
receive signals from the at least one ultrasound transducer array corresponding to the imaging of the intracranial vessels of a patient;
perform one or more image processing techniques on the received signals; and
generate one or more high-resolution intravascular images of the intracranial vessels of the patient based on the one or more image processing techniques.

27. The neurovascular ultrasonic system of claim 26, wherein the one or more image processing techniques utilize artificial intelligence for advanced image reconstructions.

28. The neurovascular ultrasonic system of claim 26, wherein the one or more image processing techniques utilize adaptive filtering techniques to reduce noise and enhance the signal-to-noise ratio.

29. The neurovascular ultrasonic system of claim 26, wherein the one or more image processing techniques utilize computational super-resolution algorithms from multiple ultrasound frames to create the one or more high-resolution intravascular images.

30. The neurovascular ultrasonic system of claim 26, wherein the one or more image processing techniques utilize doppler processing to display blood flow information.

31. The neurovascular ultrasonic system of claim 26, wherein the imaging system is configured to perform the one or more image processing techniques on the received signals to determine neurovascular structures, one or more medical abnormalities in the patient, and one or more neurological diagnostic information associated with the intracranial vessels of the patient.

32. The neurovascular ultrasonic system of claim 26, wherein the one or more medical abnormalities include at least one of strokes, tumor localization and characterization, detection of cerebral aneurysms, intracranial atherosclerotic disease, intracranial atherosclerotic disease, abnormal blood vessel, neurodegenerative diseases, brain injury, and brain infections.

33. The neurovascular ultrasonic system of claim 26, wherein the one or more neurological diagnostic information includes at least one of monitoring intracranial pressure (ICP), guidance of brain biopsies, effects of medical treatments, guiding the delivery of the therapeutic agent, guidance of clot removal, aneurysm planning and repair guidance, tumor ablation guidance, deep brain simulation, guidance of stem cell therapies, monitoring effects of hypothermia therapy, and guidance of placement of shunts.

34. The neurovascular ultrasonic system of claim 26, wherein the generated one or more high resolution intravascular images of the intracranial vessels of the patient are displayed, wherein the intravascular images include at least cross-sectional and/or longitudinal views, of vessel wall, lumen, blood flow information, and/or 3D reconstructions of neurovascular structures.

35. A method for imaging neurovascular structures within a patient's brain using a neurovascular ultrasonic catheter, the method comprising:
accessing an arterial or venous system of the brain through a radial or femoral access;
positioning the neurovascular ultrasonic catheter in a region of interest, wherein the neurovascular ultrasonic catheter having a longitudinal axis, a proximal end, and a distal end, and the neurovascular ultrasonic catheter comprising:
at least one ultrasound transducer array integrated at the distal end of the neurovascular ultrasonic catheter, wherein the at least one ultrasound transducer array is configured for neuro intravascular ultrasound imaging of intracranial vessels;
at least one catheter shaft disposed between the distal end and the proximal end and having at least one additional lumen for insertion of a guidewire or therapeutic agent delivery; and
one or more electrical interconnects within the neurovascular ultrasonic catheter configured for electrically coupling the neurovascular ultrasonic catheter with an imaging system to transmit signals corresponding to the neuro intravascular ultrasound imaging of the intracranial vessels,
wherein the at least one ultrasound transducer array is positioned side-facing relative to the longitudinal axis of the at least one catheter shaft and offset to the distal end of the neurovascular ultrasonic catheter; and
utilizing the at least one ultrasound transducer array at the distal end of the neurovascular ultrasonic catheter to image stenosis, blood clots, wall thickness of aneurysms, tumors, and other malformations.

36. The method of claim 35, wherein the distal end of the neurovascular ultrasonic catheter is tapered at an angle to facilitate ease of navigation through the intracranial vessels of the patient, wherein the distal end is made of materials acoustically matching with blood, the materials include a thermoplastic polyurethane (TPU), thermoplastic elastomer (TPE) (e.g., PEBA for example, available under the trade name PEBAX®), or a synthetic polymer (e.g., silicone).

37. The method of claim 35, wherein the at least one ultrasound transducer array corresponds to a micro-electro-mechanical systems (MEMS) ultrasound transducer array.

38. The method of claim 35, wherein the at least one ultrasound transducer array is configured for phased array imaging or linear array imaging or a combination of phased array imaging and linear array imaging of the intracranial vessels of the patient.

39. The method of claim 35, wherein the at least one ultrasound transducer array comprises piezoelectric micromachined ultrasonic transducers (PMUTs).

40. The method of claim 35, wherein the at least one ultrasound transducer array comprises capacitive micromachined ultrasonic transducers (CMUTs).

41. The method of claim 35, wherein the at least one ultrasound transducer array comprises epitaxial lead zirconate titanate (Epi-PZT) thin film substrate.

42. The method of claim 35, wherein the at least one ultrasound transducer array is made from a piezocomposite or a single-crystal piezoelectric.
